(19) United States
(12) Patent Application Publication
(10) Pub. No.: US 2026/0201459 A1
(43) Pub. Date: Jul. 16, 2026

(54) SYSTEMS AND METHODS FOR SEQUENCING NUCLEIC ACIDS FROM SINGLE EXTRACELLULAR VESICLES

(71) Applicant: University of Florida Research Foundation, Incorporated, Gainesville, FL (US)

(72) Inventors: Thomas D. SCHMITTGEN, Gainesville, FL (US); Thomas Ettor ANGELINI, Gainesville, FL (US); Andrew BROCK, Gainesville, FL (US); Jinmai JIANG, Gainesville, FL (US); Senthilkumar DURAIVEL, Gainesville, FL (US)

(21) Appl. No.: 19/133,884

(22) PCT Filed: Nov. 30, 2023
(86) PCT No.: PCT/US2023/081836

§ 371 Date: May 29, 2025

Related U.S. Application Data
(60) Provisional application No. 63/385,858, filed on Dec. 2, 2022.

(51) Int. Cl. C12Q 1/6869; C12N 5/00; C12Q 1/48; C12Q 1/6844; C12Q 1/6876; C40B 70/00

(52) U.S. Cl. CPC C12Q 1/6869; C12N 5/0062; C12N 5/0075; C12Q 1/48; C12Q 1/6844; C12Q 1/6876; C12Y 207/07049; C40B 70/00; C12N 2513/00; C12N 2533/30; C12N 2830/50; C12Q 2600/112

(57) ABSTRACT

Described are systems, compositions, and methods for printing droplets containing single extracellular vesicles. The systems, compositions, and methods can be used to perform a biochemical reaction, such as a sequencing reaction, on a single extracellular vesicle.

### CROSS-REFERENCE TO RELATED APPLICATIONS

This application claims the benefit of U.S. Provisional Application No. 63/385,858, filed Dec. 2, 2022, which is incorporated herein by reference.

### BACKGROUND

Extracellular vesicles (EVs) are nanosized vesicles that are released from nearly all cell types. EVs encompass both exosomes (40-120 nm in diameter) which are secreted from multivesicular bodies and microvesicles (50-1,000 nm) produced through direct budding of the plasma membrane. EVs play important roles in many physiological processes including coagulation, inflammatory response, cell maturation, adaptive immune response, bone calcification and neural cell communication (Yanez-Mo M et al. “Biological properties of extracellular vesicles and their physiological functions. J Extracell Vesicles.” 2015; 4:27066). EVs also play roles during cancer progression such as suppression of immune surveillance in the tumor microenvironment and establishment of the premetastatic niche. EVs have been identified in nearly all biofluids and contain a variety of cargo including mRNA, miRNA, long and short noncoding RNAs, proteins and lipids. Due to their stability in the bloodstream and ability to transfer their contents to cells in the body (Valadi H et al. “Exosome-mediated transfer of mRNAs and microRNAs is a novel mechanism of genetic exchange between cells.” Nat Cell Biol. 2007; 9(6):654-9), there is broad translational potential for EVs in oncology, for example as tumor biomarkers.

An impediment to the development of EVs as cancer biomarkers is the vast heterogeneity in vesicle structure, composition, content and function (FIG. 1) (Nieuwland R et al. “Reproducibility of extracellular vesicle research.” Eur J Cell Biol. 2022; 101(3):151226.). EV heterogeneity is often masked as most studies of the functional significance of vesicles have investigated EVs in bulk and used ensemble-averaging assays (i.e., ‘omics’) for data interpretation (Bordanaba-Florit G et al. “Using single-vesicle technologies to unravel the heterogeneity of extracellular vesicles.” Nat Protoc. 2021; 16(7):3163-85). Bulk approaches to vesicle characterization cannot address the heterogeneity of tumor-derived EVs.

Single-vesicle analysis approaches have been developed in an attempt to address three fundamental questions in regards to characterizing EV heterogeneity: (i) subpopulations, membrane protein and lipid composition and vesicle content, (ii) assays to probe the metabolic activity of EV proteins and (iii) characterization of the EV content and function depending on the cells of origin (Bordanaba-Florit et al. 2021). Existing single-vesicle analysis include Nanoparticle Tracking Analysis (NTA), cryo-TEM, high resolution flow cytometry, surface-enhanced Raman spectroscopy (SERS), atomic force microscopy, super-resolution microscopy and digital droplet PCR to name a few. Other examples of single-vesicle analysis assays include combining microfluids and antibody-based detection to study the surface proteins of individual EVs (Wu D et al. “Profiling surface proteins on individual exosomes using a proximity barcoding assay.” Nat Commun. 2019; 10(1):3854) and immunocapture of isolated EVs for determining protein (Lee K et al. “Multiplexed Profiling of Single Extracellular Vesicles.” ACS Nano. 2018; 12(1):494-503; Liu C et al. “Single-Exosome-Counting Immunoassays for Cancer Diagnostics.” Nano Lett. 2018; 18(7):4226-32) or lipid (Smith Z J et al. “Single exosome study reveals subpopulations distributed among cell lines with variability related to membrane content.” J Extracell Vesicles. 2015; 4:28533; Lee et al. 2018; Liu et al. 2018) composition. These techniques fail to address certain fundamental questions regarding EV heterogeneity, characterizing the entirety of the nucleic acid contents (e.g., mRNA, miRNA, small and large noncoding RNAs) of EVs at single vesicle resolution.

Microfluidic technologies have been developed for the formation of droplets at the instant that a single cell enters a fluidic orifice, creating “drops on demand” containing single cells (Franke T et al. “Surface acoustic wave actuated cell sorting (SAWACS).” Lab Chip. 2010; 10(6):789-94.; Chen A et al. “On-chip magnetic separation and encapsulation of cells in droplets.” Lab Chip. 2013; 13(6):1172-81.; Collins D J et al. “Surface acoustic waves for on-demand production of picoliter droplets and particle encapsulation.” Lab Chip. 2013:13(16):3225-31; Collins D J et al. “The particle valve: On-demand particle trapping, filtering, and release from a microfabricated polydimethylsiloxane membrane using surface acoustic waves” Applied Physics Letters. 2014:105; Schmid L et al. “Sorting drops and cells with acoustics: acoustic microfluidic fluorescence-activated cell sorter.” Lab Chip. 2014; 14(19):3710-8; Brouzes E et al. “Rapid and continuous magnetic separation in droplet microfluidic devices.” Lab Chip. 2015; 15(3):908-19). Other methods of generating droplets containing a single cell leverage the statistics of low numbers, where continuous streams of droplets are produced from cell dispersions at extremely low cell concentrations. Poisson statistics are leveraged in which 95% of droplets are empty but 98% of cell-containing droplets contain just a single cell (Clausell-Tormos J et al. “Droplet-based microfluidic platforms for the encapsulation and screening of Mammalian cells and multicellular organisms.” Chem Biol. 2008; 15(5):427-37; Seemann R et al. “Droplet based microfluidics.” Rep Prog Phys. 2012; 75(1):016601; Lagus T P et al. “A review of the theory, methods and recent applications of high-throughput single-cell droplet microfluidics.” Journal of Physics D: Applied Physics. 2013; 46(11)). In both of these approaches to single-cell encapsulation, sophisticated microfluidic devices are designed and controlled, and interfacial phenomena between the aqueous and organic fluids must be carefully controlled by tuning fluid viscosities and specialized surfactant concentrations while simultaneously controlling fluid-wetting phenomenon at the walls of the microfluidic devices (Grunera P et al. “Stabilisers for water-in-fluorinated-oil dispersions: Key properties for microfluidic applications.” Current Opinion in Colloid & Interface Science. 2015; 20(3):183-91).

At the nanoscale, microfluidic technology has been hampered by low throughput and susceptibility to clogging of the nanofluidics (Ko J et al. “Combining Machine Learning and Nanofluidic Technology To Diagnose Pancreatic Cancer Using Exosomes.” ACS Nano. 2017: 11(11):11182-93). Moreover, the microfluidics systems are unable to detect the 100 nm EVs. Current systems designed to create droplets containing a single cell will not work for EVs.

### SUMMARY

Described are methods of forming droplets containing a single cell or extracellular vesicle and a single microbead comprising: forming an aqueous suspension containing cells or extracellular vesicles; and three-dimensional (3D) printing the aqueous suspension into a yield-stress support medium, wherein the 3D printing forms isolated droplets of the aqueous suspension in the yield-stress support medium. The extracellular vesicle can be, but is not limited to, an exosome or a microvesicle. In some embodiments, the aqueous suspension further comprises microbeads. The microbead can be, but is not limited to, a DNA barcoded microbead. In some embodiments, greater than 90% of the droplets contain 0, 1, or 2 cells or extracellular vesicles and 0, 1, or 2 microbeads. In some embodiments, ≥65% of droplets contain 1 bead; ≥25% of the droplets contain 1 cell or extracellular vesicle; or ≥15% of the droplets contain 1 microbead and 1 cell or extracellular vesicle.

In some embodiments, the aqueous suspension comprises the extracellular vesicles at a concentration of 10-60,000 extracellular vesicles/μL and the microbeads at a concentration of 1900-120,000 microbeads/μL. In some embodiments, the aqueous suspension comprises the extracellular vesicles at a concentration of 100 to about 500 extracellular vesicles/μL.

In some embodiments, the aqueous suspension further contains one or more reagents to perform a biochemical reaction. The biochemical reaction can be, but is not limited to: a nucleic acid synthesis reaction, a poly adenylation reaction, a sequencing reaction, or a nucleic acid amplification reaction. In some embodiments, the nucleic acid amplification reaction comprises PCR or RT-PCR. The one or more reagents can include one or more of dNTPs, DTT. ATP, poly(A) polymerase, RNase inhibitor, reaction buffer, DNA polymers, reverse transcriptase, salt, buffer, and a surfactant (e.g., a detergent).

In some embodiments, the yield-stress support medium comprises a jammed organic microgel. The jammed organic microgel can be, but is not limited to, self-assembled block copolymer particles swollen with an organic solvent. The yield-stress support medium can be formed by mixing a mass of one or more block copolymers into a volume of an organic solvent. In some embodiments, the yield-stress support medium is hydrophobic. In some embodiments, the yield-stress support medium is immiscible with the aqueous suspension.

In some embodiments, neither the aqueous suspension nor the yield-stress support medium comprises a surfactant. In some embodiments, the aqueous suspension comprises a surfactant and the yield-stress support medium does not comprise a surfactant.

In some embodiments, 3D printing comprises disposing the aqueous suspension through an inner lumen of a printing nozzle into the yield-stress support medium, wherein the printing nozzle is dimensioned and configured to cause formation of the droplet of the aqueous suspension being disposed therethrough. In some embodiments, the inner lumen is in fluid communication with a supply comprising the aqueous suspension. The printing nozzle can have an inner radius of from about 75 μm to about 375 μm. In some embodiments, printing nozzle has an inner diameter that is between about 0.10% and about 10% greater than a diameter of the microbead. In some embodiments, the inner diameter of the printing nozzle is between about 0.1% and about 10% larger than an outer diameter of the droplet.

The aqueous suspension can be disposed through the printing nozzle at a flow rate of from about 1 to about 300 μL/hr. In some embodiments, the aqueous suspension is disposed through the printing nozzle at a flow rate of about 1 μL/hr to about 135 μL/hr. In some embodiments, the aqueous suspension is disposed through the printing nozzle at a flow rate of about 3 μL/hr to about 25 μL/hr. In some embodiments, the aqueous suspension is disposed through the printing nozzle at a flow rate of about 3 μL/hr. In some embodiments, the aqueous suspension is disposed through the printing nozzle at a flow rate of about 25 μL/hr. In some embodiments, the aqueous suspension is disposed through the printing nozzle at a flow rate of about 3, 4, 5, 6, 7, 8, 9, 10, 11, 12, 13, 14, 15, 16, 17, 18, 19, 20, 21, 22, 23, 24, or 25 μL/hr.

In some embodiments, the printing nozzle is translated through the yield-stress support medium during the disposing. The printing nozzle can be translated through the yield-stress support medium at a rate of from about 1 to about 30 mm/sec. In some embodiments, the printing nozzle is translated through the yield-stress support medium at a rate of about 10 to about 20 mm/sec. In some embodiments, the printing nozzle is translated through the yield-stress support medium at a rate of about 20 mm/sec. In some embodiments, the printing nozzle is translated through the yield-stress support medium at a rate of about 10 mm/sec. In some embodiments, the printing nozzle is translated through the yield-stress support medium at a rate of about 10, 11, 12, 13, 14, 15, 16, 17, 18, 19, or 20 mm/sec.

Described is a three-dimensional (3D) printing apparatus comprising: a printing nozzle comprising a lumen formed along a longitudinal axis of the printing nozzle and having an inner diameter; at least one processor; and at least one memory storing program codes, wherein the at least one memory and the program codes are configured, with the at least one processor, to cause the 3D printing apparatus at least to: dispose droplets of an aqueous suspension through the printing nozzle into a yield-stress support medium, wherein respective droplets of said aqueous suspension are disposed in an isolated location in the yield-stress support medium, and wherein the aqueous suspension contains cells or extracellular vesicles and microbeads. In some embodiments, the one or more extracellular vesicles comprise an exosome or a microvesicle. The microbeads can be, but are not limited to, DNA barcoded microbeads. The yield-stress support medium can be, but is not limited to, a jammed organic microgel. The jammed organic microgel can be, but is not limited to, a self-assembled block copolymer particles swollen with an organic solvent. In some embodiments, the yield-stress support medium is immiscible with the aqueous suspension. In some embodiments, greater than 90% of the droplets contain 0, 1, or 2 cells or extracellular vesicles and 0, 1, or 2 microbeads. In some embodiments, ≥65% of droplets contain 1 bead; ≥25% of the droplets contain 1 cell or extracellular vesicle; or ≥15% of the droplets contain 1 microbead and 1 cell or extracellular vesicle.

In some embodiments, the 3D printing apparatus further comprises a mixer configured to mix a mass of one or more block copolymers with a volume of an organic solvent to form the yield-stress support medium.

In some embodiments, the 3D printing apparatus further comprises a reservoir configured to store a supply of the aqueous suspension, wherein the at least one memory and the program codes are further configured, with the at least one processor, to cause the 3D printing apparatus at least to communicate a portion of the aqueous suspension from the supply through the inner lumen of the printing nozzle, the printing nozzle being dimensioned and configured to cause formation of the droplet of the aqueous suspension being communicated therethrough.

In some embodiments, the 3D printing apparatus further comprises a conduit configured to communicate said one or more extracellular vesicles into the printing nozzle. The conduit can be dimensioned and configured to communicate exactly one exosome into the barcoded hydrogel microsphere once disposed within the droplet and before the droplet is disposed within the yield-stress support material.

In some embodiments, an inner diameter of the printing nozzle of the 3D printing apparatus is between about 0.10% and about 10% larger than an outer diameter of the droplet.

Described are methods of performing a biochemical reaction on the contents of a single cell or extracellular vesicle comprising forming one or more droplets each containing a single cell or extracellular vesicle and a single microbead and performing the biochemical reaction within the one or more droplets. In some embodiments, the method further comprises lysing the cell or extracellular vesicle within the droplet. Lysing the cell or extracellular vesicle can comprise heating the yield-stress support medium containing the droplets to about 50° C. In some embodiments, the cell or extracellular vesicle is lysed, such as by heating the yield-stress support medium containing the droplets to about 50° C., and incubated for a period of time and under conditions appropriate to perform the biochemical reaction. In some embodiments, the method further comprises separating the yield-stress support medium and the droplets. Separating the yield-stress support medium and droplets can comprise centrifuging the yield-stress support medium and the droplets contained in the yield-stress support medium. Centrifuging the yield-stress support medium containing the droplets may or may not result in fusion of the droplets. In some embodiments, the method further comprises pooling the droplets.

In some embodiments, the microbead comprises a DNA barcoded microbead. In some embodiments, the microbead comprises a DNA barcoded microbead and the biochemical reaction comprises a DNA synthesis reaction and/or a DNA sequencing reaction. In some embodiments, the method further comprises exposing the droplets to conditions appropriate to release barcoded cDNA primers from the microbeads (e.g., exposure to 350 nm light). In some embodiments, the method further comprises performing a DNA amplification reaction. A DNA sequencing reaction can be used to sequence one or more DNAs or RNAs of the single cell or extracellular vesicle. In some embodiments, the biochemical reaction further comprises adding a poly A tail to at least one RNA present in the cell or extracellular vesicle. In some embodiments, the biochemical reaction does not comprise adding a poly A tail to any RNAs present in the cell or extracellular vesicle.

Described are methods of sequencing one or more RNAs present in a plurality of individual exosomes comprising:
(a) forming a plurality of droplets each containing a single exosome and a single DNA barcoded microbead, wherein the individual bead of the plurality of beads are spatially separated (e.g., 3D printed) in a yield-stress support medium;
(b) heating the yield-stress support medium containing the droplets to about 50° C., thereby lysing the exosomes;
(c) exposing the droplets to 350 nm light, thereby releasing barcoded cDNA primers from the microbeads;
(d) performing a reverse transcriptase reaction, wherein reverse transcriptase present in the droplet reverse transcribes the one or more RNAs present in the exosomes using primers released from the DNA-barcoding microbeads thereby forming cDNAs;
(e) centrifuging the yield-stress support medium and the droplets contained in the yield-stress support medium, thereby separating the yield-stress support medium from the droplets and collecting the droplets;
(f) performing a sequencing reaction on the cDNAs.

The one or more the RNAs can be, but are not limited to, mRNA, small and large non-coding RNA, miRNA, tRNA, rRNA, fragments of rRNA, and tRNA and combinations thereof.

In some embodiments, the methods further comprise performing a DNA amplification reaction prior to step (f), thereby amplifying the cDNAs of step (d). In some embodiments, the methods further comprise adding a poly(A) tail to one or more of the RNAs prior to step (d).

Described are methods of analyzing a sample from a subject comprising: sequencing RNAs from extracellular vesicles, thereby identifying RNAs expressed by one or more cell types in the subject. Identifying RNAs expressed by one or more cell types in the subject is used to diagnose a disease.

### BRIEF DESCRIPTION OF THE DRAWINGS

FIG. 1. Diagram illustrating release of EVs with heterogeneous cargo from tumor cells (bottom) and normal cells (top).

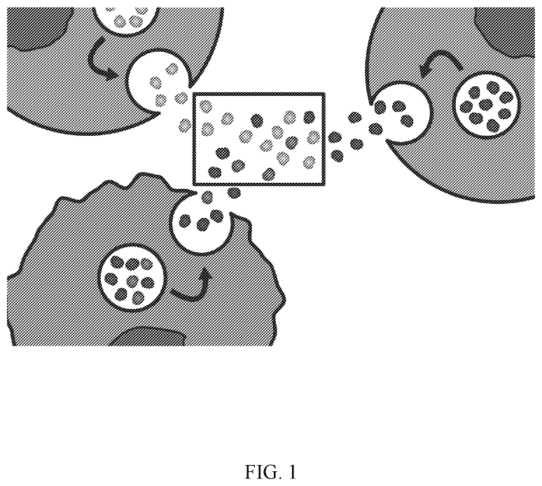

FIG. 2. Schema for single EV sequencing. (a) Schematic of single EV transcriptome barcoding in drops. A solution containing EVs, barcoded hydrogel beads, and reverse transcriptase is printed into a microgel such that a percentage of the printed droplets contain one EV and one barcoded hydrogel bead. After EV and hydrogel bead encapsulation, the barcoding cDNA primers are released from the beads using >350 nm UV light, followed by EV lysis at 50° C., poly(A)+ RNA capture (optional), and reverse transcription. The aqueous and organic phases are separated by centrifugation. The cDNA contained within the aqueous phase is sequenced using whole transcriptome sequencing. (b) Schematic of barcoding cDNA primers attached to hydrogel beads.

FIG. 3. 3D printing as a platform for biochemical reactions. (A) Image showing pattern of 1060 rhodamine droplets and collected epifluorescence microscopy images demonstrating generation of 3D print arrays of droplets. (B) Enlarged section of image in (A) showing isolated droplets. (C) Image showing separation of aqueous phase from hydrocarbon-based gel by centrifugation. An aqueous solution of bromophenol blue was pipetted into a sample of microgel material and then centrifuged. (D) Image of printed droplets. Arrows indicate the presence of single fluorescent microspheres as a model of exosomes. (E) Image of an electrophoresis gel showing nucleic acid amplified from RT-PCR reactions performed within 3D printed droplet arrays. Aqueous and gel components were separated by centrifugation as in (C). (lane 1=Amplicon of 18S rRNA, lane 2=no reverse transcription control, lane 3=cDNA positive control that was not printed, and lane 4=no template control PCR)

FIG. 4. Graph illustrating printing optimization of fluorescent beads. Probability of printing different numbers of fluorescently labeled beads per printed droplet.

FIG. 5. EV printing conditions to optimize droplet loading. (A) Printing conditions were found such that nearly all EV-containing droplets contain one 63 μm BDP conjugated bead. (B) Confocal image of a single green-labeled EV (arrows) within a droplet containing one BDP conjugated bead (red). (C) Graph illustrating the probability of printing zero, one or ≥2 EVs per droplet as determined by manual counting of about 100 confocal images of droplets. Images collected with 60× objective.

FIG. 6. qRT-PCR of the RNA contents of printed exosomes. CD9(+), CD63(+) & CD81(+) exosomes with a mean diameter of 100 nm (A) were purified from THP-1 cells using antibody based immunocapture. Approximately 3.500 exosomes were printed into the hydrogel. oligo dT labeled beads were used to prime the poly(A)+ RNA, followed by cDNA synthesis in the individual printer droplets of the gel. The aqueous phase containing the cDNA was collected into a reaction tube for subsequent workup including an overnight in vitro transcription (IVT). (B) Agilent Bioanalyzer graph illustrating results from the IVT reveals the presence of poly(A)+ RNA ranging in size from 200 to 6,000 nt (average 3,537 nt). The cDNA synthesized from the IVT RNA was assayed by qRT-PCR primed with either oligo dT or stem loop specific primers to the mature miRNA. (C) Table showing the presence of the eukaryotic translation elongation factor EEF1A1, GAPDH and FTL but the absence of the mature miRNAs.

FIG. 7. Single EV sequencing library preparation.

FIG. 8. Workflow and hypothetical results for single EV data. (A) Graph illustrating quality control via examination of summary statistics, e.g., the total number of genes and reads per EV. (B) Data pre-processing steps like normalization and multiplet detection. (C) Comparison of single EV data to bulk EV data. (D) Unsupervised analysis to find EV clusters. (E) Differential expression and annotation of identified clusters.

FIG. 9A-B. Organoid model of early pancreatic cancer development. (A) Under stressful conditions such as inflammation, normal pancreatic acini transdifferentiate into ductal cells by acinar to ductal metaplasia (ADM). This process is reversible; however, when challenged by a mutation in KRASG12D, ADMs will irreversibly progress to PanIN and invasive PDAC. (B) Experimentally, primary human pancreatic acinar cells obtained from deceased organ donors will transdifferentiate over 6 days of culture. Acinar cells will be infected with a lentivirus expressing mutant KRASGI2D. EVs will be collected from the media and their contents sequenced to discover EVs that are associated with KRASG12D.

FIG. 9C. Organoid model of early pancreatic cancer development. (C) Human acinar cells transdifferentiated to ductal cells by day 6 when they were stained with the viability dye Calcein AM.

FIG. 10. Single vesicle sequencing identifies rare EVs. (A) Shown are hypothetical EVs that are released only from KRASG12D positive cells (diamond) or those secreted by both KRASG12D or control cells (circles). The RNA contents are depicted by different colored lines within each EV. (B) Bulk RNA sequencing analysis of the EVs will not provide a true estimate of the RNA's heterogeneity obscuring any diagnostic value. (C) Single EV analysis would reveal underlying heterogeneity and detect rare events (yellow).

FIG. 11. Graphs illustrating RNA reads per barcode and coverage for sequencing poly(A+) luciferase mRNA. (A) For a sample of luciferase RNA, the distribution of unique reads per barcode is displayed after restricting to barcodes with at least 20 reads. Among this set of barcodes, 90% of captured reads corresponded to luciferase. (B) captured reads were aggregated across barcodes to examine coverage of the luciferase transcript. The coverage value corresponds to the number of reads spanning the given position after read alignment.

FIG. 12. Graphs illustrating RNA reads per barcode and coverage for sequencing a small, poly(A⁻) Hy5 RNA. (A) For a sample of Hy5 RNA, the distribution of unique reads per barcode is displayed after restricting to barcodes with at least 20 reads. Among this set of barcodes, 90% of captured reads corresponded to Hy5 RNA. (B) captured reads were aggregated across barcodes to examine coverage of the Hy5 transcript. The coverage value corresponds to the number of reads spanning the given position after read alignment.

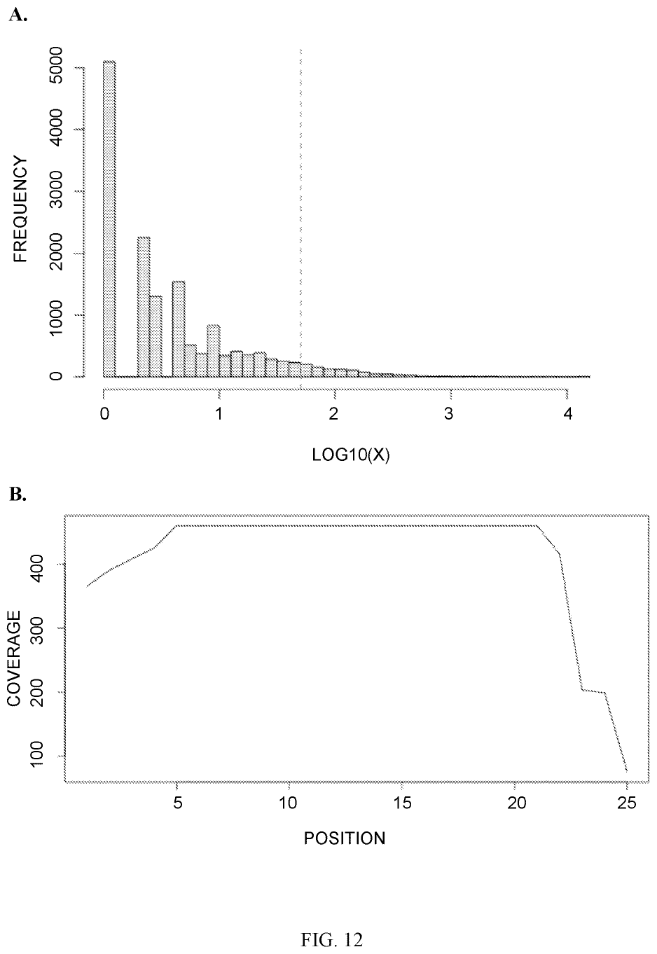

### DETAILED DESCRIPTION

### I. Definitions

Before describing the present teachings in detail, it is to be understood that the disclosure is not limited to specific compositions or process steps, as such may vary. As used in this specification and the appended claims, the singular form “a,” “an,” and “the” include plural references unless the context clearly dictates otherwise. Thus, for example, reference to “a peptide” includes a plurality of peptides and the like. The conjunction “or” is to be interpreted in the inclusive sense, i.e., as equivalent to “and/or,” unless the inclusive sense would be unreasonable in the context.

The use of “comprise.” “comprises,” “comprising,” “contain,” “contains,” “containing,” “include.” “includes.” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The term “about” or “approximately” means within an acceptable error range for the particular value as determined by one of ordinary skill in the art, which will depend in part on how the value is measured or determined, i.e., the limitations of the measurement system. For example. “about” can mean within 1 or more than 1 standard deviation, per the practice in the art. Alternatively, “about” can mean a range of up to 0 to 20%, 0 to 10%, 0 to 5%, or up to 10% of a given value. Where particular values are described in the application and claims, unless otherwise stated the term “about” meaning within an acceptable error range for the particular value should be assumed. In the context of the lengths of nucleotide sequences, the terms “about” or “approximately” are used these lengths encompass the stated length with a variation (error range) of 0 to 10% around the value (X±10%).

All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

“Bioprinting” comprises precise deposition of bio-ink (e.g., aqueous suspension) containing cells or cell components (e.g., extracellular vesicles), optionally with one or more additional components. The deposition of the bio-ink can be in two or three dimensions. Bioprinting can be automated or semi-automated. Bioprinting can be computer controlled or aided. Bioprinting can be performed using a bioprinter.

A bioprinter dispenses a bio-ink in two or three dimensions. A bioprinter can dispense the bio-ink in precise amounts in a particular geometry by moving a printer head or nozzle relative to a printer stage or receiving substrate adapted to receive bio-ink (e.g., a sacrificial support medium or yield-stress support medium). In some embodiments, a bioprinter achieves a particular geometry by moving a printer stage or receiving substrate relative to the printer head or nozzle.

A bioprinter comprises a means for dispensing bio-ink through a printer nozzle. A bioprinter can comprises at least one reservoir adapted to contain the bio-ink. The reservoir is in fluid communication with the printer nozzle. In some embodiments, a bioprinter dispenses the bio-ink via application of application of a piston, application of pressure, application of compressed gas, application of hydraulics, or application of a combination thereof such that the bio-ink is dispensed through the printer nozzle.

A bioprinter can be adapted to dispense the bio-ink in predefined unit volumes and/or at a predefined flow rate.

A bioprinter can be adapted to maintain the bio-ink at a predefined temperature. In some embodiments, a bioprinter is adapted to dispense the bio-ink at a predefined temperature.

In some embodiments, bioprinting involves using a computer to configure parameters such as printer nozzle position and bio-ink dispense rate or volume. In some embodiments, a computer is used to specify the direction and speed of the movement (translation) of the printer nozzle. The position and translation of a printer nozzle can be controlled in one or more of the x, z, and z axes. Position and translation of the printer nozzle can be control using any means available in the art for controlling the printer nozzle of a 3D printer.

Membranous vesicles are released by a variety of cells into the extracellular microenvironment. Based on the mode of biogenesis, these membranous vesicles can be classified into three broad classes (i), extracellular vesicles (EVs, also termed exosomes) (following the MISEV 2018 guideline, the term EVs is used to cover exosomes as a specific subpopulation of membranous vesicles that includes exosomes, and excludes ectosomes (microvesicles) and apoptotic bodies), (ii), ectosomes or microvesicles, and (iii) apoptotic bodies. Extracellular vesicles are cell-derived vesicles originating from endosomal compartments produced during the vesicular transport from the endoplasmic reticulum (ER) to the Golgi apparatus. Extracellular vesicles are released extracellularly after the multivesicular bodies are fused with the plasma membrane. Extracellular vesicles are distinct from both ectosomes and apoptotic bodies in size, content, and mechanism of formation. Ectosomes are vesicles of various size (typically 0.1-1 mm in diameter) that bud directly from the plasma membrane and are shed to the extracellular space. Ectosomes have on their surface the phospholipid phosphatidylserine. Apoptotic bodies are formed during the process of apoptosis and engulfed by phagocytes.

“Subject” refers to an animal, such as a mammal, for example a human. The methods described herein can be useful in both humans and non-human animals. In some embodiments, the subject is a mammal (such as an animal model of disease), and in some embodiments, the subject is human.

### II. 3D Printed Droplets

Described are systems, compositions, and methods for efficiently forming droplets containing a single cell, extracellular vesicle (exosome), ectosome, microvesicle, exomeres, supermeres, or apoptotic body and a single microbead. The droplets are formed by 3D printing an aqueous suspension containing the cells, extracellular vesicles, ectosomes, microvesicles, or apoptotic bodies and the microbeads into a support medium (e.g., a sacrificial support medium or yield-stress support medium). In some embodiments, the droplets are formed by 3D printing an aqueous suspension containing the exosomes and the microbeads into a support medium.

Described are systems and methods that utilize 3D printing to creating shapes from fluid phases and trap them stably in a 3D space. The systems and methods comprise 3D printing cells, exosomes, ectosomes, microvesicles, or apoptotic bodies within aqueous droplets in a microgel-based support medium. In some embodiments, the droplets further comprise a microbead. In some embodiments, greater than 90% of the droplets contain 0, 1, or 2 cells or extracellular vesicles and 0, 1, or 2 microbeads. In some embodiments. ≥65% of droplets contain 1 bead; ≥25% of the droplets contain 1 cell or extracellular vesicle; or ≥15% of the droplets contain 1 microbead and 1 cell or extracellular vesicle

The 3D printing can be performed using any 3D bioprinter or 3D bioprinting device suitable for use with the aqueous suspension (bio-ink) containing the cells extracellular vesicles, ectosomes, microvesicles, or apoptotic bodies and the microbeads. In some embodiments, 3D printing comprises extrusion 3D bioprinting.

The 3D bioprinter deposits the aqueous suspension as droplets into the support medium, wherein the droplets are suspended in the support medium. The support medium provides physical confinement of the droplets during the printing process such that each printed droplet is isolated from the other printed droplets. In some embodiments, the printing nozzle is translated through the yield-stress support medium during the printing.

The described systems, compositions, and methods can be used to form (print) droplets containing a single cell, extracellular vesicle, ectosome, microvesicle, or apoptotic body and a single microbead, wherein the droplet is about 50 to about 150 μm in diameter. In some embodiments, the printed droplets are about 50 to about 100 μM in diameter. In some embodiments, the printed droplets are about 60 to about 80 μm in diameter. In some embodiments, the printed droplets are about 60, about 65, about 70, about 75, or about 80 μm in diameter. In some embodiments, the printed droplets are 605, 65±5, 70±5, 75±5, or 80±5 μm in diameter. In some embodiments, the printed droplets are about 70 μm in diameter. In some embodiments, the printed droplets are 70±1, 70±2, 70±3, 70±4, or 70±5 μm in diameter.

The described systems, compositions, and methods can be used to print droplets containing a single cell, extracellular vesicle, ectosome, microvesicle, or apoptotic body and a single microbead in a support medium, wherein the droplets are spaced about 100 to about 500 μm apart. In some embodiments, the droplets are printed in the support medium about 100, about 120, about 140, about 160, about 180, about 200, about 220, about 240, about 260, about 280, about 300, about 320, about 340, about 360, about 380, about 400, about 420, about 440, about 460, about 480, or about 500 μm apart. In some embodiments, the droplets are printed in the support medium about 140 μm apart. In some embodiments, the droplets are printed in the support medium 140±1, 140±2, 140±3, 140±4, 140±5, 140±6, 140±7, 140±8, 140±9, or 140±10 μm apart. The droplets can be printed in the support media in a single layer on in multiple layers. In some embodiments, the droplets are printed in the support media in 1, 2, 3, 4, 5, 6, 7, 8, 9, or 10 or more layers.

The described systems, compositions, and methods can be used to print droplets containing a single cell, extracellular vesicle, ectosome, microvesicle, or apoptotic body and a single microbead in a support medium, wherein the droplets are printed using a nozzle translation speed of about 1 mm/s to about 30 mm/sec. In some embodiments, the nozzle translation speed is about 1, about 2, about 3, about 4, about 5, about 6, about 7, about 8, about 9, about 10, about 12, about 14, about 16, about 18, about 20, about 22, about 24, about 26, about 28, or about 30 mm/sec. In some embodiments, the nozzle translation speed is about 10 mm/sec. In some embodiments, the nozzle translation speed is about 20 mm/sec. In some embodiments, the nozzle translation speed is about 10, 11, 12, 13, 14, 15, 16, 17, 18, 19, or 20 mm/sec.

The described systems, compositions, and methods can be used to print droplets containing a single cell, extracellular vesicle, ectosome, microvesicle, or apoptotic body and a single microbead in a support medium, wherein the bio-ink is dispensed at a volumetric flow rate of about 1 μL/h to about 300 μL/h. In some embodiments, the bio-ink is dispensed at a volumetric flow rate of about 1, about 2, about 3, about 4, about 5, about 6, about 7, about 8, about 9, about 10, about 12, about 14, about 16, about 18, about 20, about 25, about 30, about 35, about 40, about 45, about 50, about 55, about 60, about 65, about 70, about 75, about 80, about 85, about 90, about 95, about 100, about 110, about 120, about 130, about 135, about 140, about 150, about 160, about 170, about 180, about 190, about 200, about 220, about 240, about 260, about 280, or about 300 μL/h. In some embodiments, the bio-ink is dispensed at a volumetric flow rate of about 1 to about 135 μL/hr. In some embodiments, the bio-ink is dispensed at a volumetric flow rate of about 1 to about 50 μL/hr. In some embodiments, the bio-ink is dispensed at a volumetric flow rate of about 1 to about 30 μL/hr. In some embodiments, the bio-ink is dispensed at a volumetric flow rate of about 3 to about 25 μL/hr. In some embodiments, the bio-ink is dispensed at a volumetric flow rate of about 3 μL/h. In some embodiments, the bio-ink is dispensed at a volumetric flow rate of about 25 μL/hr.

The described systems, compositions, and methods can be used to print droplets containing a single cell, extracellular vesicle, ectosome, microvesicle, or apoptotic body and a single microbead in a support medium, wherein the inner diameter of the bioprinter nozzle is about 50 μm to about 375 μm. In some embodiments, the inner diameter of the bioprinter nozzle is about 50, about 55, about 60, about 65, about 70, about 75, about 80, about 85, about 90, about 95, about 100, about 110, about 120, about 130, about 140, about 150, about 160, about 170, about 180, about 190, about 200, about 220, about 240, about 260, about 280, about 300, about 325, about 350, or about 375 μm. In some embodiments the nozzle is selected to have an inner diameter of about the size of any microbeads used in the bio-ink. In some embodiments the nozzle is selected to have an inner diameter of the corresponds to about 100% to about 150% of the diameter of any microbeads used in the bio-ink. In some embodiments the nozzle is selected to have an inner diameter of the corresponds to about 100% to about 140% of the diameter of any microbeads used in the bio-ink. In some embodiments the nozzle is selected to have an inner diameter of the corresponds to about 100% to about 130% of the diameter of any microbeads used in the bio-ink. In some embodiments the nozzle is selected to have an inner diameter of the corresponds to about 100% to about 120% of the diameter of any microbeads used in the bio-ink. In some embodiments the nozzle is selected to have an inner diameter of the corresponds to about 100% to about 110% of the diameter of any microbeads used in the bio-ink. In some embodiments, the inner diameter of the bioprinter nozzle is about 65 to about 70 μm.

In some embodiments, the concentration of cells, extracellular vesicles, ectosomes, microvesicles, or apoptotic bodies and microbeads in the bio-ink, the nozzle translation speed, the volumetric flow rate, and nozzle diameter are selected to print droplets wherein about 60% to about 100% of the droplets contain a single microbead and about 15 to about 30% or more of the droplets containing a single microbead also contain a single cell, extracellular vesicle, ectosome, microvesicle, or apoptotic body. In some embodiments, the concentration of cells, extracellular vesicles, ectosomes, microvesicles, or apoptotic bodies and microbeads in the bio-ink, the nozzle translation speed, the volumetric flow rate, and nozzle diameter are selected to print droplets wherein about 15%, about 16%, about 17%, about 18%, about 19%, about 20%, about 21%, about 22%, about 23%, about 24%, about 25%, about 26%, about 27%, about 28%, about 29%, or about 30% or more of the droplets contain a single microbead and a single extracellular vesicle. In some embodiments, the concentration of cells, extracellular vesicles, ectosomes, microvesicles, or apoptotic bodies and microbeads in the bio-ink, the nozzle translation speed, the volumetric flow rate, and nozzle diameter are selected to print droplets wherein about 17% or more of the droplets contain a single microbead and a single extracellular vesicle.

In some embodiments, biochemical reactions can be performed within the printed droplets. The biochemical reactions can be, but are not limited to, reverse transcription, polyadenylation. DNA amplification, and nucleic acid sequencing.

### A. Bio-Ink

Described are compositions comprising aqueous suspensions of cells or exosomes and microbeads that can be used as bio-inks for used in 3D bioprinting. Also described are compositions comprising aqueous suspensions of ectosomes, microvesicles, apoptotic bodies and microbeads that can be used as bio-inks for used in 3D bioprinting.

In some embodiments, the microbead comprises a barcoded microbead. The barcoded microbead can be, but is not limited to, a DNA barcoded microbead. In some embodiments, each DNA barcoded microbead in the bio-ink is uniquely barcoded so that each droplet and its contents are distinguishable. In some embodiments, the microbead comprises a hydrogel microbead. The DNA barcoded microbead can be any commercially available DNA barcoded microbead. The DNA barcoded microbead can also be any previously described DNA barcoded microbead, including, but not limited to the DNA barcoded microbeads described in international patent publications WO2016040476. WO2018075693, WO2017139690, and WO2017096158, each of which is incorporated herein by reference.

In some embodiments, the microbeads are about 25 to about 100 μm in diameter. In some embodiments the microbeads are about 40 to about 80 μm in diameter. In some embodiments, the microbeads are about 50, about 55, about 60, about 65, about 70, about 75, or about 80 μm in diameter. In some embodiments, the microbeads are 50±5, 55±5, 60±5, 65±5, 70±5, 75±5, or 80±5 μm in diameter. In some embodiments, the microbeads are about 60 to about 70 μm in diameter. In some embodiments, the microbeads are about 60, about 61, about 62, about 63, about 64, about 65, about 66, about 67, about 68, about 69, or about 70 μm in diameter.

The cell and be any cell. The cell can be a bacteria cell, an archaea cell, or a eukaryotic cell. The eukaryotic cell can be a mammalian cell, such as, but not limited to, a human cell. The human cell can be from a subject. The exosome, ectosome, microvesicle, or apoptotic body can be from any cell capable of producing exosomes, ectosomes, microvesicles, or apoptotic bodies. The exosome, ectosome, microvesicle, or apoptotic body can be from, for example, a cell, a culture cell, a biofluid, plasma sample, a clinical specimen, a biopsy sample, a laboratory sample, or an environmental sample. In some embodiments, the exosome, ectosome, microvesicle, or apoptotic body is from a mammalian cell or subject. The cells, exosomes, ectosomes, microvesicles, or apoptotic bodies can be from a sample obtained from a mammalian subject. The mammalian subject can be, but is not limited to, a human subject. The sample can be, but is not limited to, a blood sample or a tissue sample. A sample may be processed. The cells, exosomes, ectosomes, microvesicles, or apoptotic bodies can be isolated and/or purified prior to addition to the bio-ink. Cells, exosomes, ectosomes, microvesicles, or apoptotic bodies from a sample can be isolated and/or purified by any means known in the art for isolating or purifying cells, exosomes, ectosomes, microvesicles, or apoptotic bodies. In some embodiments, exosomes are purified using differential ultracentrifugation, optionally with density gradient purification, immunocapture methods, sequential centrifugation, centrifugation using a sucrose density gradient or sucrose cushion, ultrafiltration, ExoQuick (System biosciences), Total Exosome Isolation Kit (Invitrogen), immunoaffinity capture, microfluidics-based isolation, and combinations thereof. In some embodiments, the cells, exosomes, ectosomes, microvesicles, or apoptotic bodies can be stored prior to 3D printing. The cells, exosomes, ectosomes, microvesicles, or apoptotic bodies can be stored at a temperature from about 4° C. to about −80° C.

The bio-ink may further comprise one or more reagents (e.g., chemicals, enzyme, buffers) for performing one or more biochemical reactions. In some embodiments, the bio-ink comprises one or more reagents for performing a reverse transcription reaction. In some embodiments, the bio-ink comprises one or more reagents for performing one or more of a polyadenylation reaction, a nucleic acid synthesis reaction, a nucleic acid amplification reaction, an in vitro transcription reaction, or a sequencing reaction. In some embodiments, the bio-ink comprises one or more reagents for performing a polyadenylation reaction and a reverse transcription reaction.

In some embodiments, the bio-ink comprises: (a) cells, exosomes, ectosomes, microvesicles, or apoptotic bodies; (b) DNA barcoded microbeads; and (c) one or more of a poly(A)polymerase, ATP, a reverse transcriptase, dNTPs, a buffer. DTT, an RNase inhibitor, and a DNA polymerase. The poly(A) polymerase can be, but is not limited to: an E. coli poly(A)polymerase or a thermostable poly(A) polymerase. The thermostable poly(A) polymerase can be, but is not limited to, a Thermus aquaticus (Taq) poly(A) polymerase. The reverse transcriptase can be, but is not limited to, a thermostable reverse transcriptase. The thermostable reverse transcriptase can be, but is not limited to, a Superscript IV thermostable reverse transcriptase, and one or more additional primers.

In some embodiments, the bio-ink (or aqueous suspension) contains a surfactant. In some embodiments, the bio-ink (or aqueous suspension) does not contain a surfactant.

### B. Sacrificial Support Medium

The support medium is configured to allow the aqueous suspension to be extruded, injected, or deposited into it as droplets at predefined positions and to support the droplets at the predefined positions. In some embodiments, the support medium comprises a sacrificial support medium. In some embodiments, the sacrificial support medium comprises a yield-stress material, such as a yield-stress fluid. In some embodiments, the yield-stress material has self-healing properties. Application of a stress to the sacrificial support medium, such as by translocation of a 3D bioprinter nozzle through the sacrificial support medium that overcomes a critical stress (the yield stress), initiates flow and renders the media of the sacrificial support medium liquid-like. After the disturbance of sacrificial support medium's microstructure by a translocating 3D bioprinter nozzle and its displacement by any deposited material (e.g., the aqueous suspension droplet), the microstructure of the sacrificial support medium spontaneously recovers. The self-healing capacity permits the medium to transition from a fluidized state back to a solid-like state, thereby encapsulating the deposited material. The stress generated by the translocating 3D bioprinter print nozzle results in localized yielding of the microgel support without disturbing previously printed constructs. The three-dimensional shape of the sacrificial support medium is not particularly limited.

In some embodiments, the yield-stress material comprises a jammed microgel. In some embodiments, the jammed microgel comprises a hydrophobic jammed organic microgel. Microparticles in a jammed system are densely packed and immobilized by physical interactions with surrounding particles, resulting in macroscopic materials that behave as solids until enough force is applied to induce movement. In some embodiments, the jammed microgel comprises a jammed organic microgel. In some embodiments, the jammed microgel is any jammed organic microgel suitable for use with 3D bioprinting. The jammed organic microgel can be, but it not limited to, jammed granular particles, entangled polymer solutions, micelles packed into solid-like phases, and polymer networks with reversible bonds. In some embodiments, the jammed organic microgel comprises self-assembled block copolymer particles swollen with an organic solvent. In some embodiments, the jammed microgel is formed by mixing a mass of one or more block copolymers into a volume of an organic solvent.

In some embodiments, the support medium does not contain a surfactant. In some embodiments, the jammed microgel does not contain a surfactant.

### III. Single Cell/Exosome Biochemical Reactions

Described are methods of performing a biochemical reaction on the contents of a single cell, exosome, ectosome, microvesicle, or apoptotic body. The biochemical reaction can be, but is not limited to, a reverse transcription reaction, a poly adenylation reaction, a nucleic acid synthesis reaction, a sequencing reaction, or combinations thereof. In some embodiments, the described methods of can be used to perform a biochemical reaction on the contents of a single exosome. In some embodiments, the described methods can be used to sequence one or more nucleic acids present in single cells, exosomes, ectosomes, microvesicles, or apoptotic bodies.

Described are methods for sequencing one or more nucleic acids present in a single cell, exosome, ectosome, microvesicle, or apoptotic body, comprising:
(a) forming an aqueous suspension comprising
   (i) cells, exosomes, ectosomes, microvesicles, or apoptotic bodies;
   (ii) DNA barcoded microbeads; and
   (iii) reagents for performing one or more of a polyadenylation reaction, a reverse transcription reaction, a nucleic acid synthesis reaction, a nucleic acid amplification reaction, an in vitro transcription reaction, and a sequencing reaction;
(b) 3D printing a droplet containing a single cell, exosome, ectosome, microvesicle, or apoptotic body and a single DNA barcoded microbead in a support medium using any of the systems, compositions, and methods described for forming such droplets, wherein the aqueous suspension is used as a bio-ink;
(c) exposing the droplet to conditions (e.g., 350 nm light) suitable for release of (e.g., cDNA) primers from the DNA barcoded microbead;
(d) heating the droplet or support medium in which the droplet is printed, to lyse the cell, exosome, ectosome, microvesicle, or apoptotic body; and
(e) incubating the droplet under conditions suitable for performing one or more of the polyadenylation reaction, the reverse transcription reaction, the nucleic acid synthesis reaction, the nucleic acid amplification reaction, the in vitro transcription reaction, the sequencing reaction, or a combination of two or more of the polyadenylation reaction, the reverse transcription reaction, the nucleic acid synthesis reaction, the nucleic acid amplification reaction, the in vitro transcription reaction, and the sequencing reaction.

In some embodiments, heating the droplet comprises heating the droplet to 50° C. In some embodiments, heating the droplet comprises heating the droplet to about 50° C. for about 2 hours.

In some embodiments, the aqueous suspension further comprises reagents for performing a reverse transcription reaction. In some embodiments, the aqueous suspension comprises reagents for performing a reverse transcription reaction and a polyadenylation reaction. In some embodiments, the reverse transcription reaction and/or a polyadenylation reaction are performed simultaneously with heating the droplet in step (d).

In some embodiments, the method further comprises heating the droplet to 80° C. following step (e) to inactivate any enzymes present in the aqueous suspension or the droplet.

In some embodiments, a reverse transcription reaction or a reverse transcription reaction and a polyadenylation reaction are performed in step (e). Polyadenylation can be used to add a poly(A) tail to the 3′ end of any poly(A)⁻ RNA present in the droplet. The reverse transcription and the polyadenylation reaction can be performed sequentially or simultaneously. Poly(A)⁺ RNA present in the droplet is primed for reverse transcription using oligonucleotide primers provided by the DNA barcoded microbeads, cDNAs of any RNAs present in the droplets is formed by the reverse transcription reaction.

In some embodiments, the generated cDNAs can be isolated and used for nucleic acid sequencing using nucleic acid sequencing methods available in the art. In some embodiments, the generated cDNAs can be isolated and amplified using nucleic acid amplification methods available in the art. The amplified nucleic acids can then be sequenced.

In some embodiments, a plurality of droplets are pooled following step (e). The 3D printed droplets can be separated from the hydrophobic support medium by centrifugation. Centrifugation of the plurality of droplets and the support medium results in separation of the aqueous (droplet) and organic (support media) phases. Following centrifugation, the aqueous phase containing the contents of the droplets, including any products of any biochemical reactions performed in the droplet, can be isolated from the support media.

In some embodiments, a reverse transcription reaction or a reverse transcription reaction and a polyadenylation reaction are performed in step (e) to form cDNA of any RNAs present in the droplets and two or more droplets are pooled following step (e). The droplets can be pooled by centrifuging the droplets in the supporting medium, whereby the droplets are separated from the support medium, and collecting the separated droplets or aqueous phase. Pooling of a plurality of droplets following reverse transcription, forms a pooled cDNA library.

In some embodiments, the pooled cDNA library is used in a sequence reaction using any nucleic acid sequencing method, such as a next gen sequencing reaction, available in the art. In some embodiments, the pooled cDNA library is used in DNA amplification reaction using any nucleic acid amplification method available in the art for amplifying cDNA. The amplified cDNA can then be sequenced.

In some embodiments, the methods comprise forming, via 3D printing, a plurality of droplets each containing a single cell, exosome, ectosome, microvesicle, or apoptotic body and a single DNA barcoded microbead: performing reverse transcription reactions in each of the plurality of droplets, wherein any RNA present in the plurality of droplets is reverse transcribed using primers from the DNA barcoded microbeads to form cDNAs containing barcoded sequences, pooling the cDNAs, and sequencing the pooled barcoded cDNAs. In some embodiments, the sequencing comprises next generation sequence. In some embodiments, the cell, exosome, ectosome, microvesicle, or apoptotic body comprises an exosome.

Reverse transcription of RNA present in the droplets, using primers from the DNA barcoding microbeads, prior to pooling of cDNA, provides for generation of barcoded cDNA, wherein any sequences can be correlated with the originating cell, exosome, ectosome, microvesicle, or apoptotic body.

The described methods can be used to perform reverse transcription reactions in each of a plurality of individual droplets to form cDNAs, wherein each cDNA formed from an RNA present in each individual droplet contains a DNA barcode from the DNA barcoded microbead present in the individual droplet. Following reverse transcription in the individual droplets, the barcoded cDNAs can be amplified and/or sequenced in bulk. The barcoding can be used in identification of the droplet (i.e., cell, exosome, ectosome, microvesicle, or apoptotic body) from whence an individual amplified sequence was derived.

In some embodiments, methods for sequencing RNAs from individual exosomes are described, the methods comprising: (a) providing a plurality of droplets, wherein each droplet of the plurality of droplets comprises (i) a single exosome; (ii) a DNA barcoded microbead; and (iii) reagents for reverse transcribing any RNAs present in the exosome; (b) performing a reverse transcription reaction in each of the plurality of droplets thereby forming barcoded cDNAs corresponding the to RNAs present the exosome of each droplet in the plurality of droplets; and (c) sequencing the barcoded cDNAs or derivatives thereof.

The systems and methods described herein provide benefits over prior technologies. Digital droplet PCR has poor throughput and can only quantify known nucleic acid entities. Micro and nanofluidic approaches are limited by the inability to detect 100 nm vesicles, poor throughput and the susceptibility to undergo clogging and agglomeration of the particles. By printing and trapping the droplets into a stable, solid-like medium, barriers and pitfalls of channel-based micro- and nano-fluidic approaches are avoided.

In some embodiments, the systems and methods can be used to sequence nucleic acids from individual cells, exosomes, ectosomes, microvesicles, or apoptotic bodies. The nucleic acids can be, but are not limited to, poly(A)⁺ and poly(A)⁻ RNA. Poly(A)⁺ includes mRNA. Poly(A)⁻ RNA includes, but is not limited to, miRNA, Y RNA, snRNA, lincRNA and fragmented mRNA, rRNA, and tRNA.

In some embodiments, the systems and methods can be used to sequence RNAs associated with single exosomes.

The described systems and methods can be used for high-throughput sequencing of nucleic acids from individual exosomes, ectosomes, microvesicles, or apoptotic bodies within a heterogeneous population of exosomes, ectosomes, microvesicles, or apoptotic bodies. The exosomes, ectosomes, microvesicles, or apoptotic bodies can be from a single cell type or from a heterogeneous cell population (e.g., multiple cell types).

Different cells release exosomes (or ectosomes, microvesicles, or apoptotic bodies) into the extracellular space that are unique in terms of their contents. In some embodiments, sequencing nucleic acids from exosomes, ectosomes, microvesicles, or apoptotic bodies can be used to identify biomarkers. In some embodiments, sequencing nucleic acids from exosomes, ectosomes, microvesicles, or apoptotic bodies can be used to identify biomarkers for evaluating health or early detection of diseases such as cancer.

In some embodiments, sequencing nucleic acids from exosomes, ectosomes, microvesicles, or apoptotic bodies can be used to identify the cell type from which the exosome, ectosome, microvesicle, or apoptotic body was formed. In some embodiments, sequencing nucleic acids from exosomes, ectosomes, microvesicles, or apoptotic bodies can be used to identify the cell type and from which the exosome, ectosome, microvesicle, or apoptotic body was formed and the disease state of the cell. In some embodiments, the sequencing nucleic acids from exosome, ectosome, microvesicle, or apoptotic body can be used to identify subjects suffering from a disease (e.g., cancer) or at risk of suffering from a disease.

In some embodiments, sequencing the RNA content of individual exosomes, ectosomes, microvesicles, or apoptotic bodies may be used to discover novel disease-specific biomarkers that may have been previously masked due to bulk RNA sequencing approaches. Data on the RNA content of individual exosomes can be used to probe fundamental questions on biological heterogeneity of exosomes. Protein positive EV populations can be isolated using immunocapture and sequenced individually to correlate RNA content to the proteomic profile. Sequencing the RNA contents of individual EVs may be used to track their cell of origin in complex biofluids such as blood or a liquid biopsy. Sequencing RNA content of individual exosomes may be used to assist in the development of EV-based therapeutics, for example by providing the quantity and distribution of the therapeutic and non-therapeutic cargo within the individual EVs.

### EXAMPLES

### Example 1. 3D Printing of EVs and Performing Single EV eDNA Synthesis

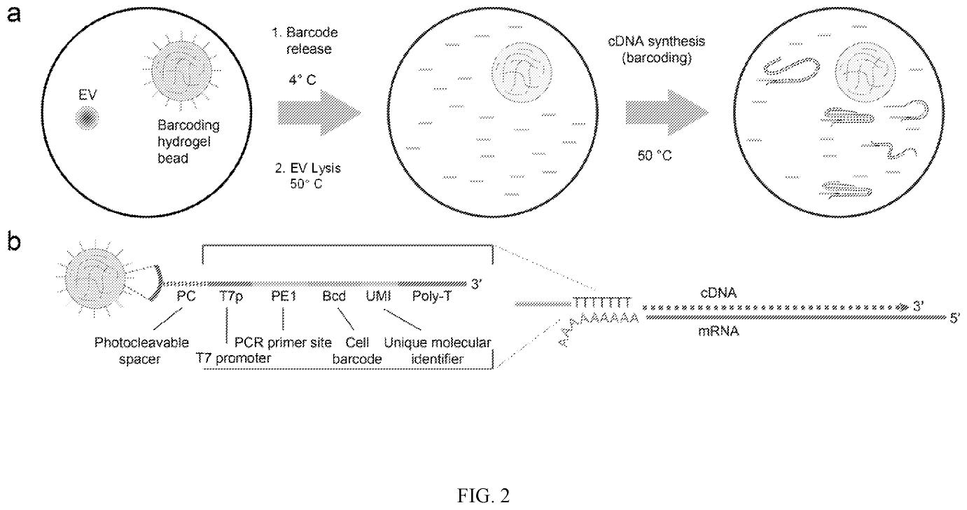

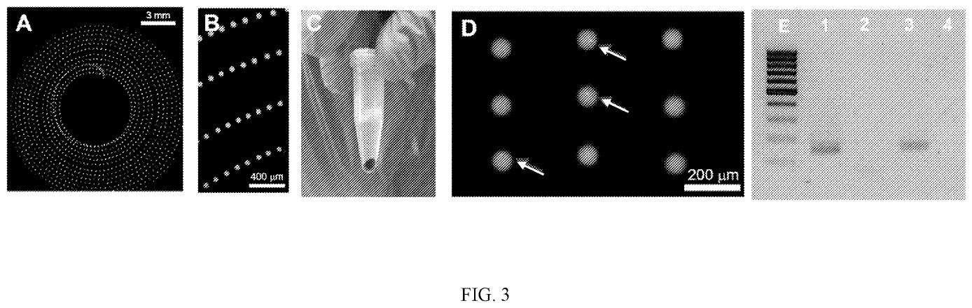

The schema for performing single EV RNA sequencing is shown in FIG. 2. Experiments were conducted to test the ability to print and perform biochemical reactions in arrays of droplets (FIG. 3). Briefly, a suspension containing EVs, barcoded hydrogel beads (RAN Biotechnologies, Beverly, MA) and biochemicals to perform reverse transcription reactions are 3D printed into droplet arrays supported by an embedding medium. This medium is made from packed microgels swollen in mineral oil (O'Bryan C S et al. “Self-assembled micro-organogels for 3D printing silicone structures.” Sci Adv. 2017; 3(5):e1602800). Printing is conducted under conditions in which the number of droplets containing one barcoded bead and one EV is maximized, while the number containing multiplets is minimized. After EV and hydrogel bead encapsulation, the barcoding cDNA primers are released from the beads using 350-nm UV light. The gel medium is heated to 50° C. to lyse the EVs and perform the reverse transcription reaction using Superscript IV thermostable reverse transcriptase (Thermo). This enzyme is active at 50° C. but can be inactivated by heating to 80° C. for 10 min of 80° C. The aqueous and organic phases are separated by centrifugation (FIG. 3C) and the cDNA contained within the aqueous phase may be used for qPCR directly or for RNA sequencing library generation.

### Example 2. 3D Printing of BDP-Conjugated Beads as Models of Bar-Coded Beads

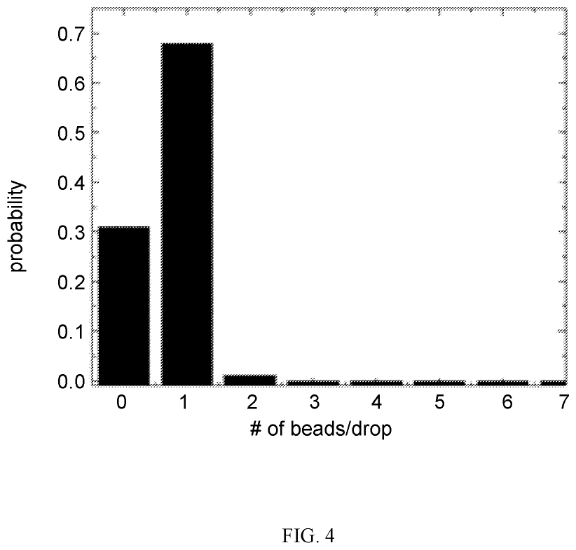

Experiments were performed to test the ability to print aqueous droplets containing controlled numbers of hydrogel beads into the packed microgel medium. BDP-conjugated beads (63 μm diameter) were purchased from RAN Biotechnologies. These beads are identical to those containing 10⁹ barcoded oligos to be used for the RNA sequencing except they are BDP dyed and do not contain the oligos. A variety of printing conditions were analyzed, varying needle type, nozzle diameter, nozzle translation speed, flow rate, and bead concentration. Following printing, the number of droplets that contain 0, 1, 2 or more beads per droplet were counted using confocal microscopy. Using the conditions shown in FIG. 4, approximately 67% of printed droplets contained one bead, 31% are empty, and the remaining 2% contain two beads.

### Example 3. 3D Printing of BDP-Conjugated Beads and EVs

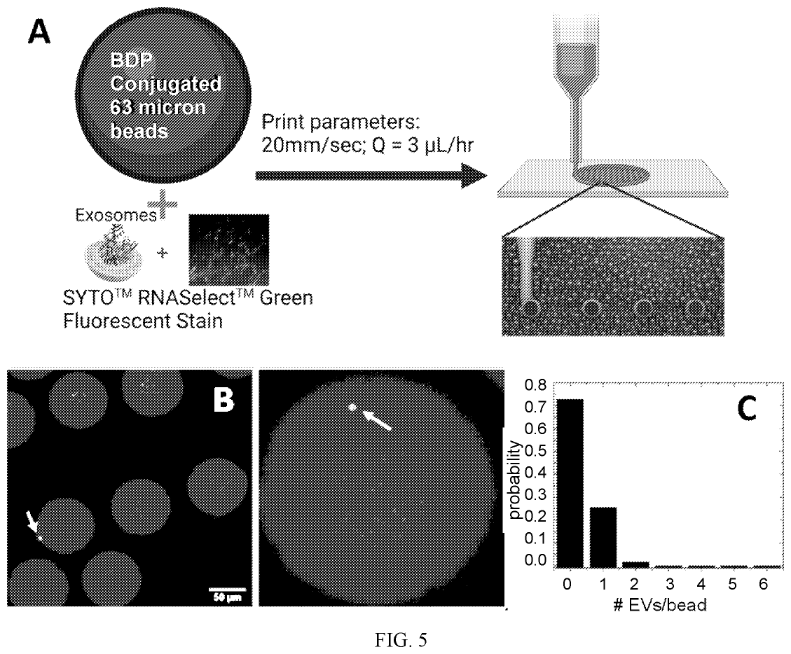

Experiments were performed to establish conditions for printing both fluorescently labeled EVs and labeled beads together, aiming to encapsulate no more than one EV per droplet. HEK293T EVs (mean diameter 100 nm) were stained with SYTO™ RNASelect™ Green Fluorescent Stain (Thermo). The BDP labeled beads and EVs were printed at 20 mm/sec at a flow rate of 3 μL/hr (FIG. 5A). Confocal microscopy captured the stained EVs along with BDP-conjugated beads that were contained within each droplet (FIG. 5B). Images of individual droplets were analyzed to determine the number of EVs in each droplet containing a bead. The statistics reported in FIG. 5C demonstrate that 25% of the droplets contained one EV and one bead. 73% contained no EVs and the remaining 2% contained 2 EVs. Based on these statistics and the current printing speed, we estimate that approximately 10⁶ droplets could be printed in two hours, with 167,500 of these [10⁶×0.67 (FIG. 4)×0.25 (FIG. 5C)] containing one oligo labeled bead and one EV. Thus, it is expected that RNA sequencing at the single EV resolution can be performed for 167,500 vesicles in 2 hours. In another experiment, printing 25 μL/h, approximately 1.4×10⁵ droplets were printed per hour. In a 2 h printing, 46,633 of these droplets (2×1.4×10⁵×0.67×0.25) are predicted to contain one oligo labeled bead and one EV (i.e., a sequenceable EV).

### Example 4. RT-PCR of cDNA Synthesized from EV RNA Contained within Printed Droplets

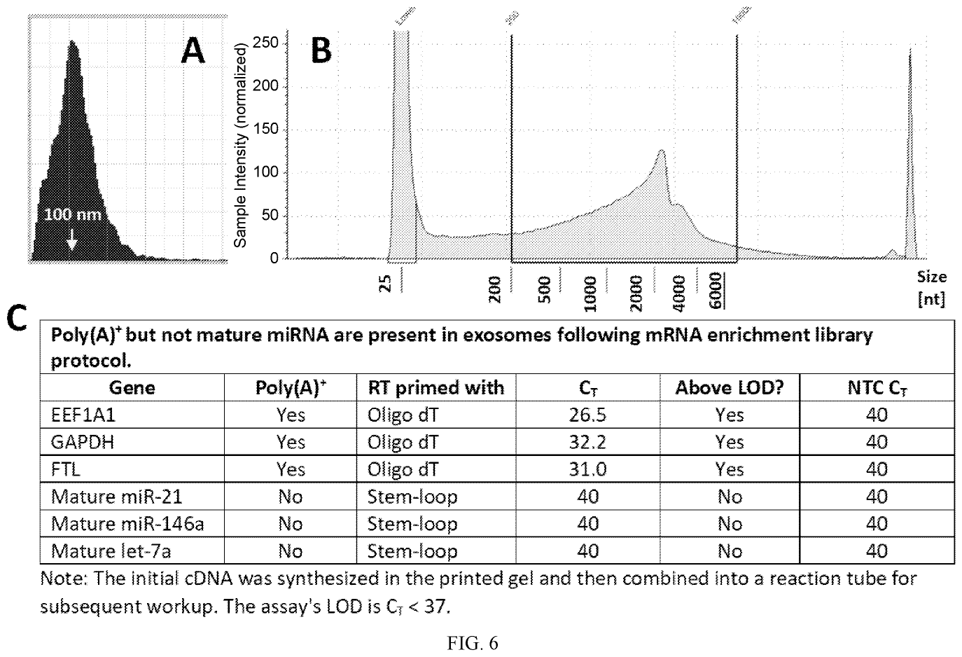

To ensure the printing and cDNA synthesis of highly pure EVs, CD63, CD81 and CD9 exosomes positive for all three markers were isolated from THP-1 monocytic leukemia cells using antibody based immunocapture (NanoPoms Lawrence, KS). The purified EVs were sized using NTA (FIG. 6A). Approximately 3.500 EVs were printed into the gel along with oligo gel beads and biochemicals to perform the cDNA synthesis including thermostable Superscript IV RT. The gel was heated to 50° C. for 2 hours to lyse the EVs and to allow the reverse transcription to complete, followed by 10 min at 80° C. to inactivate the RT enzyme. The aqueous and organic phases were separated and the aqueous phase containing the cDNA was worked up using the procedure described in FIG. 2 (and as described in Zilionis R et al. “Single-cell barcoding and sequencing using droplet microfluidics.” Nat Protoc. 2017; 12(1):44-73). Following the overnight in vitro transcription (IVT) the resulting RNA was assayed on the Agilent Bioanalyzer to estimate the length of the poly(A)+ RNA that was captured from the exosomes. The electropherograms showed the presence of intact poly(A)+RNA of 200 to 6,000 nts in length (average 3,537 nt: FIG. 6B). Following a second RT primed with oligo dT for the poly(A)+ RNA and stem-loop primers for mature miRNA, the cDNA was assayed by qPCR using gene specific primers. The CT of the EEF1A1, GAPDH and FTL were above the assay's limit of detection demonstrating their presence in the exosomes while mature miRNA was not detected under the conditions of the RNA purification (FIG. 6C). The results demonstrate that cDNA synthesized in the printed microgel from the immunoaffinity captured exosomes was successful. Collectively data demonstrate that (i) the components to perform RNA sequencing may be uniformly printed into organic microgels; (ii) the printing conditions may be leveraged such that 17% of the printed droplets contain both one oligo labeled bead and one EV; and (iii) cDNA derived from poly(A)+ RNA may be subsequently synthesized using this technology.

### Example 5. Transforming 3D Printing into a Single EV Sequencing Technology

RNA sequencing of single EVs using the described 3D printing system will be performed. Droplets of an aqueous solution of EVs, barcoded oligonucleotide beads, and biochemicals will be printed into a support material made from jammed microgels. Poly(A)+RNA will be primed using commercially available barcoded oligonucleotides linked to gel beads and the resulting cDNA will be sequenced using standard RNA sequencing methods. Preliminary experiments will be performed using fluorescent gel beads of similar composition and size to those containing the oligonucleotides. The barcoded oligonucleotide-linked beads or fluorescent gel beads will be printed along with fluorescently labeled EVs. Printing conditions varied to ensure droplet encapsulation of oligo labeled beads and EVs. mRNA loaded lipid nanoparticles and Extracellular RNA Communication Consortium (ERCC) mRNA spike mix will be used as pre-validation benchmarks. qPCR and RNA sequencing will be used as a readout to confirm that the cDNA synthesis conditions have been properly optimized. Printing conditions can be adjusted to print up to 1 million sequenceable EVs per day.

A. Rapid and controlled droplet production and EV encapsulation. To optimize printing protocols, instrument components, and material formulations capable of rapid and controlled droplet production, test prints similar to those described in FIG. 4 will be performed. Various parameters will be changed, and the size and number of droplets produced within a given printing run will be analyzed. Nozzle translation speed, v, will be varied between 1 mm/s and 30 mm/s; volumetric flow-rate, Q, will be varied between 1 μL/h and 300 μL/h; nozzle radii (inner), R, will be varied between 75 μm and 375 μm. Five increments of each parameter will be tested, creating a total of 125 different combinations. Droplet sizes will be measured with either epifluorescence or confocal fluorescence microscopy, analyzed with custom written image analysis software. In “medium-throughput” tests, between 100 and 104 droplets will be produced in each printing procedure, enabling the efficient determination of the scaling relationships that control droplet size and spacing. Parameter combinations that produce the high packing-density of droplets of the desired size (70 μm diameter) will be determined. Additional microgel concentrations which will shift fluid shear stresses at any given set of printing parameters, allowing another orthogonal degree of freedom for controlling droplet size and spacing will also be analyzed. It is anticipated that printing parameters will produce 70 μm diameter droplets spaced center-to-center by 140 μm. Producing ten layers of drops at this density on a 113 mm×76 mm plate, approximately 1-1.5×10⁶ droplets can be created in a single sample within 2 hours (Q=135 μL/h) translating the printing nozzle at 20-30 mm/s. Additional experiments will determine the concentration of oligo-labeled beads and EVs required to achieve the desired statistical distributions. Initially, tests will be performed using fluorospheres. Subsequence tests will use EVs.

Reducing the size of the oligo beads may increase the number of droplets suitable for printing. Labeled (e.g., blue) fluorospheres (diameter 100-200 nm) will be suspended in a green fluorescein solution with the BDP conjugated 63 μm beads at varying ratios and total concentrations. All droplets will be identified from the fluorescein signal in images collected using fluorescence microscopy. Integrated intensity measurements from red and blue fluorescence within these droplets will be used to quantify the number of BDP conjugated beads and blue fluorospheres within each droplet.

TABLE 2
Optimization parameters

| | translation | | | | | | |
|---|---|---|---|---|---|---|---|
| | speed | | flow rate | | droplets | Printed Droplets ^{A} | Sequenceable |
| | v | | Q | | printed/2 h | y | Single EVs ^{B} |
| phase 1 | 20 | mm/s | 3 | μL/h | 33,400 | ~5,600 | ~28,000 |
| phase 2 | 10 | mm/s | 25 | μL/h | 278,000 | ~47,000 | ~250,000 |
| Phase 3 | 1-30 | mm/s | 1-135 | μL/h | 1,500,000 | ~250,000 | ~1,260,000 |

^{A} contain 1 EV and 1 barcoded oligo: (Sequenceable EV = y × 0.67 × 0.25)
^{B} at 5 printings per day

B. Pre-validation benchmarks. As a benchmark for the initial validation, lipid nanoparticles (LNPs) loaded with codon-optimized full-length mRNA encoding firefly luciferase (luciferase-LNPs) will be used. The 1929 nt mRNA transcript will be modified with full substitution of 5-Methoxy-U, capped using CleanCap AG polyadenylated (120A). The mRNA will be formulated into LNPs using microfluids. Physiochemical properties of LNPs such as size, zeta-potential, morphology. mRNA encapsulation efficiency and mRNA copies/LNP will be determined by Dynamic Light Scattering, Nanoparticle Tracking Analysis, TEM, ribogreen assay and qPCR. Typical yields for similar sized mRNAs and comparable nanoparticles range from 2-6 copies/particle based on computational modeling (Carrasco M J et al. “Ionization and structural properties of mRNA lipid nanoparticles influence expression in intramuscular and intravascular administration.” Commun Biol. 2021: 4(1):956). Luciferase-LNPs will be printed using the procedures described above. The sensitivity and specificity of the single EV RNA sequencing platform will be determined by comparing the Luciferase copies per LNP as determined by single EV RNA sequencing to the known average of copies/LNP as determined by qPCR. As a second validation, the ERCC RNA Spike-In Mix (Thermo) along with the oligo labeled beads and biochemicals for the cDNA synthesis will be 3D printed. The ERCC RNA Spike-In Mix is a collection of 92 different poly(A)+ RNA transcripts that range from 250 to 2,000 nt in length and are intended to mimic natural eukaryotic mRNAs. By printing the spike-in solution at different known total RNA contents and mixture proportions, features of the data generating process, such as the transcript capture efficiency, length bias, and dynamic range of measurable expression at different true RNA levels will be determined. The accuracy and variability of the pipeline by direct comparison with the known transcript concentrations in the spike-in mixtures will also be determined (as described in Zheng G X et al. “Massively parallel digital transcriptional profiling of single cells.” Nat Commun. 2017; 8:14049).

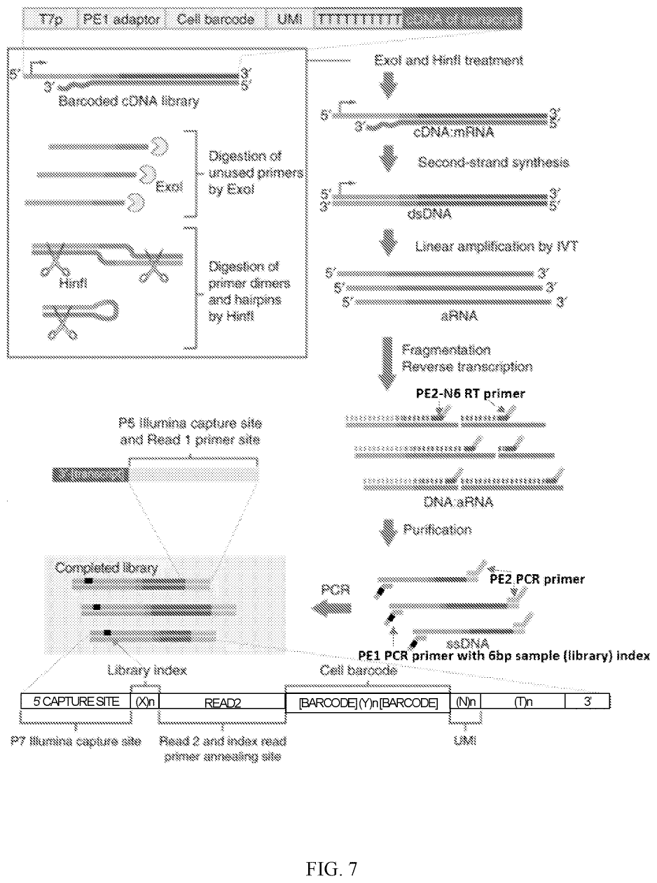

C. cDNA synthesis and library preparation from 3D printed microgels. The data shown in FIG. 7 demonstrate the ability to synthesize cDNA within droplets suspended in the microgel using the barcoded oligos bound to the hydrogels as the primer for the reverse transcription reaction. The barcoded hydrogels are 63 μm diameter beads, each containing 10⁹ barcoded oligos per bead. The gel beads were designed such that the oligos on the specific gel bead had a unique bar code to link the reads back to the individual droplet or EV and each of the 10⁹ oligos on the gel bead had a unique molecular identifier (UMI) linking the reads back to the original transcript. The oligo labeled beads are commercially available from RAN Biotechnologies (Beverly, MA). The barcoded gel beads are available in lots of one million individually barcoded gel beads. The gel beads were designed such that the oligos on the specific gel bead has a unique bar code to link the reads (sequenced nucleic acids) back to the individual droplet or EV and each of the 10⁹ oligos on the gel bead has a unique molecular identifier (UMI) linking the reads back to the original transcript. The cDNA synthesis step used for our technology as well as library preparation has been adapted from the RAN Biotechnologies published protocols for single cell poly(A)+ RNA sequencing (Zilionis et al. 2017). poly(A)+ RNA was selected as an initial proof-of-concept for the single EV sequencing technology because the commercially available RAN Biotechnologies platform is based on poly(A)+ RNA sequencing (Zilionis et al. 2017). A 50 μl suspension of approximately 17,500 HEK293T cell EVs, barcoded hydrogel beads, dNTPs, DTT, RNase inhibitor, reaction buffer and Superscript IV thermostable reverse transcriptase will be prepared. Ten μl (about 3,500 EVs) of the suspension will be printed into an aluminum chamber cast mold containing about 2 ml of the microgel. After encapsulation of the suspension into droplets, the barcoding cDNA primers are released from the beads using 350-nm UV light. Next the incubation temperature is increased to 50° C. for 2 hours to lyse the EVs, capture poly(A)+ RNA, and perform reverse transcription (FIG. 2). After generating barcoded cDNA from single EVs, 15 μl of water is added to the microgel and the printed aqueous droplets are pooled together and isolated from the microgel by centrifugation at 1500×g for 15 min. The hydrogel beads are then removed using a nucleic acid purification column (pore size 0.45 μm) with the flow-through fraction being collected into a 1.5 ml DNA LoBind tube by centrifugation at 4° C. for 1 min at 16,000×g. The detailed steps of library preparation, beginning with enzymatic cleanup of the reverse transcription reaction product are shown in FIG. 7. The 3′-5′ exonuclease ExoI is used to digest unused ssDNA primers along with the dsDNA-specific restriction endonuclease HinfI to cleave the T7 promoter sequence in primer dimers and hairpins. The library is protected from HinfI and ExoI digestion, since it is in the form of a cDNA-poly A RNA hybrid with the T7 promoter sequence as ssDNA. After a 30 min digestion at 37° C., sample purification beads (NEB) are used to isolate the cDNA-poly(A)+ RNA hybrid library followed by second-strand synthesis. Second-strand synthesis generates dsDNA that's used in an in vitro transcription reaction using T7 RNA polymerase to linearly amplify the cDNA library. The resulting amplified RNA is fragmented by zinc-ion-catalyzed cleavage, and a second reverse transcription reaction followed by limited-cycle PCR is used to convert the amplified RNA into a dsDNA library with P5 and P7 Illumina capture and primer sites that are compatible with Illumina sequencing platforms. The second reverse transcription reaction uses a random hexamer PE2-N6 RT primer to introduce a PE2 PCR hybridization start site. This library preparation step includes two quality control steps. In preparation for sequencing, barcoded libraries will be sized twice on the Agilent Bioanalyzer I) post-in vitro transcription, amplified RNA will be run on the BioAnalyzer RNA Pico assay and ii) completed library quality will be assessed by running a BioAnalyzer DNA HS assay. A diagnostic qPCR will be performed on each sample preparation to determine the correct number of cycles for PCR amplification of libraries. As detailed in FIG. 7, a sample index (6 bp library index in sequencing primer) is added during the final PCR to allow pooling of multiple libraries for multiplexed sequencing in a single run by using different PE1/PE2 primer mix variants to introduce unique Illumina library indices per sample. Individual samples will be pooled equimolarly at 1.0 nM. This “working pool” will be used as input in the NovaSeq6000 instrument sample preparation protocol. Typically, a final loading concentration of 200 pM library resulted in the best balance between sequencing data yield and quality. Under these conditions a 2×100 cycles run on an S4 flow cell should generate >2.5 billion reads/lane with ≥80% of bases at higher than Q30. Up to 50 RNA-Seq barcoded libraries will be pooled for sequencing in multiplex on a single NovaSeq 6000 S4 flow cell lane, using a 2×100 cycles (paired-end configuration), for an average of about 50 million reads pairs per sample.

D. Optimization of sequencing platform using HEK293T cell EVs. HEK293T cells will be cultured in exosome depleted fetal bovine serum and the EVs released into the culture media will be purified using two different techniques: (a) differential ultracentrifugation (Thery C et al. “Isolation and characterization of exosomes from cell culture supernatants and biological fluids.” Curr Protoc Cell Biol. 2006: Chapter 3:Unit 3 22) followed by iodixanol density gradient purification or (b) immunocapture using magnetic beads coated with antibodies specific to common exosome plasma membrane proteins CD63, CD81 and CD9 (NanoPoms, Clara Biotech Lawrence. KS). This will allow comparison of the RNA contents of highly pure exosomes isolated by immunocapture to those exosomes, microvesicles and perhaps RNPs isolated by ultracentrifugation/density gradient purification. Following purification, EVs collected from both methods will be stored at −80° C. until RNA sequencing is performed. HEK293T cell EVs will be printed under the conditions described above from a suspension containing the EVs, oligo barcoded beads (RAN Biotechnologies) and biochemicals to perform the reverse transcription reaction as described. Following the printing, the residual (unprinted) EV/biochemical suspension will be used to synthesize cDNA in a reaction tube using the identical procedure as the printed EVs. In this manner, we can directly compare the cDNA synthesized in the tube to the printed reaction. Using qPCR, the expression of 25-30 genes that are present in both the printed and unprinted (i.e., reaction tube) cDNA will be directly compared. The correlation between printed and unprinted cDNA will be determined using scatter plots. Should the correlation meet the stated performance measure (i.e., R ≥0.60), then we will proceed to the experiment described below, otherwise additional optimization will be performed.

Next, head-to-head comparison of bulk versus single EV RNA sequencing will be performed. A suspension containing the EVs, oligo barcoded beads and reverse transcription biochemicals will be prepared to be printed into the microgel. Following the printing, the residual EV/biochemical suspension in the reaction tube will be sequenced in bulk using the identical sequencing protocol as the printed EVs. The correlation between printed and unprinted bulk cDNA will be demonstrated using scatter plots.

It is expected that about 67% of the printed droplets will contain one oligo bead, of which 25% of these will also contain one EV and that printing a minimum of 10⁶ droplets in two hours gives the ability to sequence 167,500 individual EVs. The RNA Biotechnologies gel beads, designed for single cell RNA sequencing, contain 10⁹ RT oligos per bead. As these beads are designed for single cell sequencing, it is highly unlikely that 10⁹ oligos are required to capture the entire RNA contents of an individual EV. Future applications may be developed to reduce the bead's size (and thus the number of oligos per bead) and/or increase the volume of the printing plate. Both modifications will allow printing of greater numbers of smaller droplets resulting in sequencing more than 167.500 EVs per 2-hour printing. Single cell RNA sequencing typically interrogates the contents of perhaps thousands of cells, with UMI-based protocols yielding thousands of mRNA reads per cell. Because EVs are approximately 100-times smaller than cells RNA is not actively synthesized in EVs, the RNA content of EVs is significantly reduced compared to cells. While estimates vary based upon cell type, EV isolation technique, etc., EVs are reported to contain about 4 attograms of total RNA per vesicle (Wei Z et al. “Coding and noncoding landscape of extracellular RNA released by human glioma stem cells.” Nat Commun. 2017; 8(1):1145) or about 1.5 femtograms of protein per EV (Sutaria D S et al. “Low active loading of cargo into engineered extracellular vesicles results in inefficient miRNA mimic delivery.” J Extracell Vesicles. 2017:6(1):1333882).

### Example 6 Single Vesicle Sequencing Technology

Modifications of the technology will be made to allow for sequencing for both the poly(A)+ and poly(A)− RNA contents of individual EVs. Bioinformatics and statistical techniques will be applied including quality control of the reads, data cleaning and preprocessing, verification of reproducibility and accuracy and clustering and classification models. The single EV RNA sequencing platform will be evaluated using known mixtures of EVs derived from cells of different tissues to trace EV cell type of origin. A precancerous, human, pancreas organoid model will be modified to express the mutant oncogene KRASG12D and the ability of the developed technology to discover individual EVs uniquely secreted by the KRASG12D expressing cells will be assessed.

A. Single EV RNA sequencing platform for poly(A)+ RNA using mixed cells. Additional evaluation and optimization of the technology will be assessed by sequencing the EVs released from different cell lines. Cell lines derived from three different solid tumors (Panc1, pancreas; HCT-116, colorectal and A549, lung) will be cultured using standard conditions including 10% exosome depleted fetal bovine serum. Known mixtures of EVs released from the three cell lines will be sequenced by single EV RNA sequencing using the procedures described above. Printing EV mixtures in different runs will be performed in order to assess the strength of batch effects.

Statistical and bioinformatics considerations: Sequencing data quality will be analyzed using canonical summary statistics which are used in RNA-seq bioinformatics pipelines. These include the read length distribution, mapping quality scores, and GC content, among others. Capture of poly(A)+ RNAs rather than poly(A)− RNAs will also be verified. Reads will be mapped to the genome in this step. After the primary quality control is completed, “preprocessing” steps will be evaluated. The most notable aspects of this are exploratory analyses of the single EV data to understand fundamental properties such as the total read and gene counts. Examination of summary statistics will allow us to differentiate singlets from multiplets, though more sophisticated techniques designed for scRNA-seq data can be employed if necessary (Wolock S L et al. “Scrublet: Computational Identification of Cell Doublets in Single-Cell Transcriptomic Data.” Cell Syst. 2019: 8(4):281-91 e9). Additionally, normalization is considered to enable comparison of different EVs. Depending on the properties of the data, an appropriate normalization approach will be selected; to start, a simple total counts normalization will be attempted. Results will be compared to those obtained via bulk EV sequencing. In the latter case, the approximate number of EVs in a bulk sample are typically known, allowing contextualization of the efficiency of the data generating process by direct comparison with bulk data. Beyond comparison of summary statistics, sequencing will be assessed by examining the gene-level correlation between our single EV and previous bulk EV results as well as between pairs of single EVs.

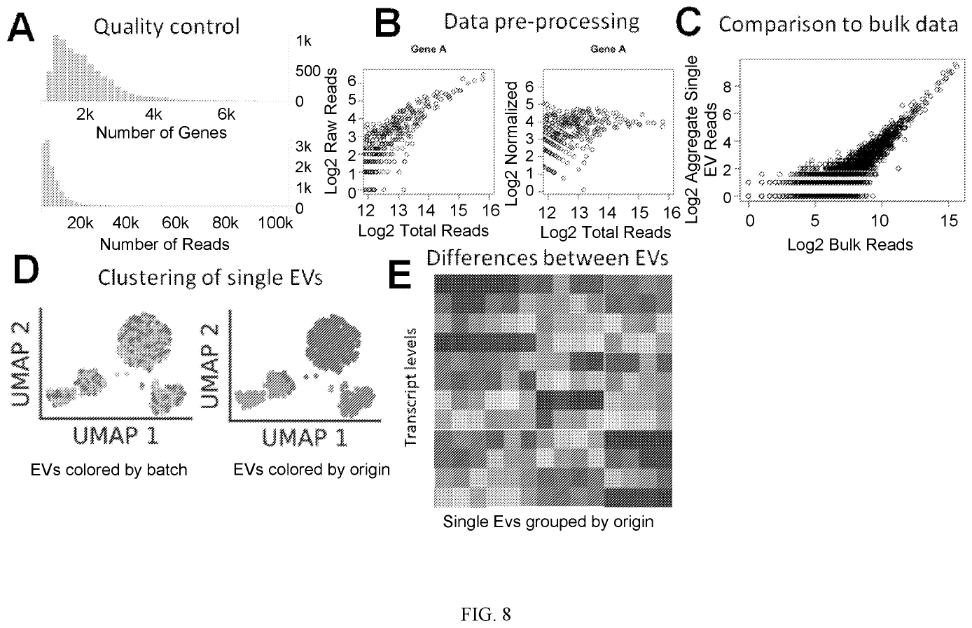

To understand the inherent heterogeneity of single EVs, “unsupervised” analyses are performed where any labels for the single EVs are not assumed. Low-dimensional embeddings of the single EVs using PCA followed by UMAP will be produced (Benegas G et al. “Robust and annotation-free analysis of alternative splicing across diverse cell types in mice.” Elife. 2022; 11). This, coupled with hierarchical clustering or similar, will reveal any natural clusterings of EVs. These clusterings are expected to correspond to the cell types of origin or subtypes therein. In scRNA-seq data, cells cluster clearly by cell type. Similar pattern using single EV sequencing are expected. Having identified natural clusters of EVs, supervised approaches will be applied to learn which factors are responsible for the separation of EVs into these clusters. The transcripts which define these clusters can be extracted via standard hypothesis testing methods for differences of means or proportions. Additional tools, such as classification models (e.g., random forests or logistic regression) will be applied to determine whether the information extracted from one experiment can be used to classify cell types of origin for EVs from a subsequent experiment. The clustered data from the first experiment will comprise the training set and be used to build the model. The data from the second experiment is then fed into the model to produce predicted cell types of origin. These predictions are compared to the clustering obtained for the new data. From this analysis, it is possible to determine how easy it is to predict the cell type of origin for a single EV that had not been seen previously, mirroring the situation in potential diagnostic applications. The bioinformatics workflow based on the method of (Benegas et al. 2022) for single EV data are shown in FIG. 8.

B. Sequencing of the total RNA content contained within single EVs. Sequencing of poly(A)+ RNAs contained within EVs will not sequence the total RNA contents of EVs including miRNA, long noncoding poly(A)− RNAs, tRNA, and rRNAs or their fragments or fragmented mRNA. To obtain a complete representation of all RNAs contained within individual EVs, a modification of the technology described above is used to sequence the poly(A)+ and poly(A)− RNA contents of the EVs. In some embodiments, a poly adenylation step to add a poly A tail to the 3′ ends of poly(A)− RNA is used. This approach has been used to prepare cDNA from mature miRNA (Shi R et al. “Facile means for quantifying microRNA expression by real-time PCR.” Biotechniques. 2005: 39(4):519-25 and Isakova A et al. “Single-cell quantification of a broad RNA spectrum reveals unique noncoding patterns associated with cell types and states.” Proc Natl Acad Sci USA. 2021; 118(51)) and long-noncoding and other noncoding RNA transcripts from single cells. As described in Isakova et al. 2021, E. coli poly(A)polymerase (New England Biolabs) and ATP can be used to add adenine tails to the 3′ prime ends of the RNA molecules. A 3D printing suspension of oligo dT barcoded hydrogel beads, RT enzyme, dNTPs, poly(A) polymerase and ATP in a reaction buffer along with the HEK293T EVs is prepared. As described above, following the 3D printing into the microgel, the contents of the droplets will be heated at 50° C. to lyse the contents of the EVs and perform the poly(A) tailing and RT simultaneously. In some embodiments, the poly(A) polymerase is a thermostable poly(A) polymerase. The thermostable poly(A) polymerase can be, but is not limited to, a Thermus aquaticus (Taq) poly(A) polymerase. The suspension will be printed into the hydrogels as described above. Following the cDNA synthesis, the aqueous and organic phases will be separated by centrifugation. cDNA synthesis for various RNAs (mature miRNA, Y RNA, lincRNA, mRNA, rRNA, tRNA, etc.) and will be validated first by qPCR and then by RNA sequencing.

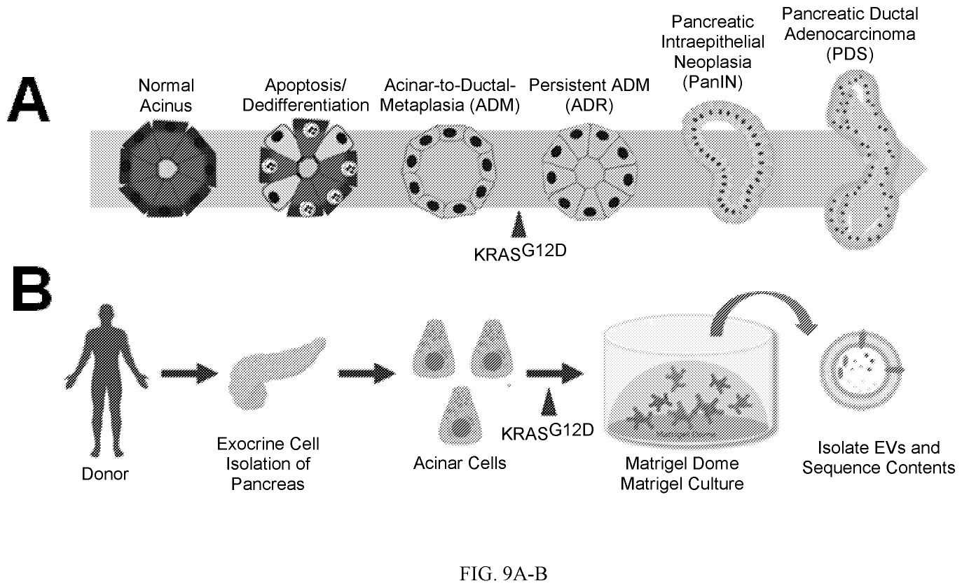

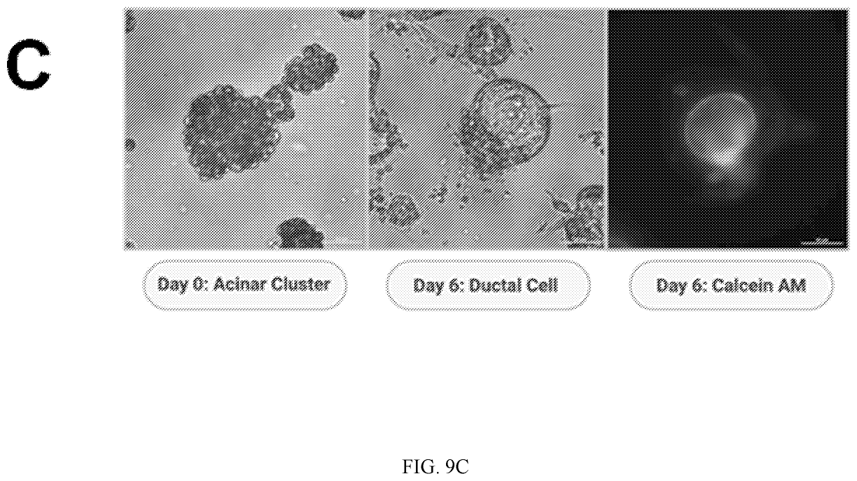

C. Discovery of EV signatures of KRAS driven PDAC as a discovery/validation system. As a second validation of the single EV sequencing platform, a pre-tumor model of pancreatic cancer will be used to discover rare EVs from pancreas organoids expressing KRASG12D. Pancreatic cancer has among the lowest 5-year survival rates due to a combination of ineffective treatments and poor means of early detection. Typically, by the time symptoms develop, the tumor is already at an advanced stage or has metastasized. Developing biomarkers that are predictive of the early events of pancreatic ductal adenocarcinoma (PDAC) development would have enormous utility. One of the earliest events in the development of PDAC is when pancreatic acinar cells transdifferentiate to ductal cells by acinar ductal metaplasia (ADM). When combined with inflammation and an activating mutation in the oncogene KRAS (e.g., KRASG12D), preneoplasia develops into invasive disease (FIG. 9A). EVs from pancreatic cancer cells expressing KRASG12D promote survival, suppress immune surveillance in the tumor microenvironment or alter the premetastatic niche. An in vitro model of acinar-to-ductal metaplasia (ADM) has been developed that: (i) recapitulates the early events of pancreatic cancer development, (ii) is a self-contained 3D organoid model that allows direct sampling of the exosomes shed into the culture media, (iii) utilizes human rather than mouse tissues and (iv) is easily genetically manipulated to express proteins of interest from lentiviral vectors.

Primary human islet-depleted pancreatic acinar cells obtained from deceased organ donors will be cultured using serum free conditions on the extracellular matrix Matrigel as described. Over 6 days of culture, the acinar cells undergo ADM to produce ductal-like epithelial cells (FIG. 9B-C). Each cell type (acinar, day 0 and ductal, day 6) is anticipated to secrete a unique repertoire of EVs into the culture media. EVs will be collected from the conditioned media daily at days 1 through 6 of the transdifferentiation and isolated using immunocapture. In this manner, day 1 conditioned media will have primarily EVs secreted from the acinar cells while day 6 media will contain primarily ductal cell secreted EVs and days 2 through 5, a mixture of EVs released from both cell types. RNA collected from the EVs on day 1 and day 6 will be sequenced in bulk to provide a baseline expression for each cell type. Single EV RNA sequencing will be performed on the EVs collected from all days. The statistical and bioinformatics analysis will be applied to determine if we can separate groups of EVs from a complex mixed cell population to determine cell of origin. To investigate the oncogene KRASG12D in the organoid model of early pancreatic cancer development, day 0 cells will be infected with a lentiviral vector to express KRASG12D while the control will be treated with empty lentivirus. Culture media will be collected daily for 6 days from the KRASG12D and control cultures. EVs will be isolated using immunocapture and sequenced by both bulk (days 1 and 6) and the single EV sequencing technology (all days). The statistical and bioinformatics analysis will be used to identify differences in RNA present in the EVs.

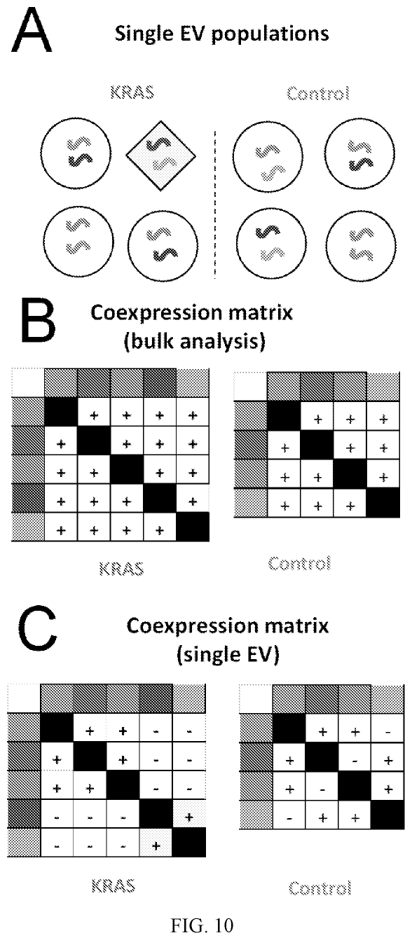

Anticipated results: Following the bioinformatics and statistical analysis of the RNA sequencing from a mixture of EVs derived from three different cell lines. UMAP plots may be used to distinguish the three groups of cell line EVs based on their cell type of origin. It is further anticipated that it will be possible to distinguish singlets from multiplets using the analysis described and that the ratios of singlets to multiplets as determined from single EV sequencing parallels those determined using the confocal microscopy imaging of printed fluorescent spheres/EVs. During ADM, the KRASG12D expressing acinar cells are expected to secrete EVs with unique RNA cargo that will distinguish them from control EVs and that such a finding may be lost using bulk RNA sequencing. Following single EV RNA sequencing, bioinformatic and statistical analysis we anticipate detecting rare events (FIG. 10A,C).

### Example 7. Sequencing in Printed Droplets

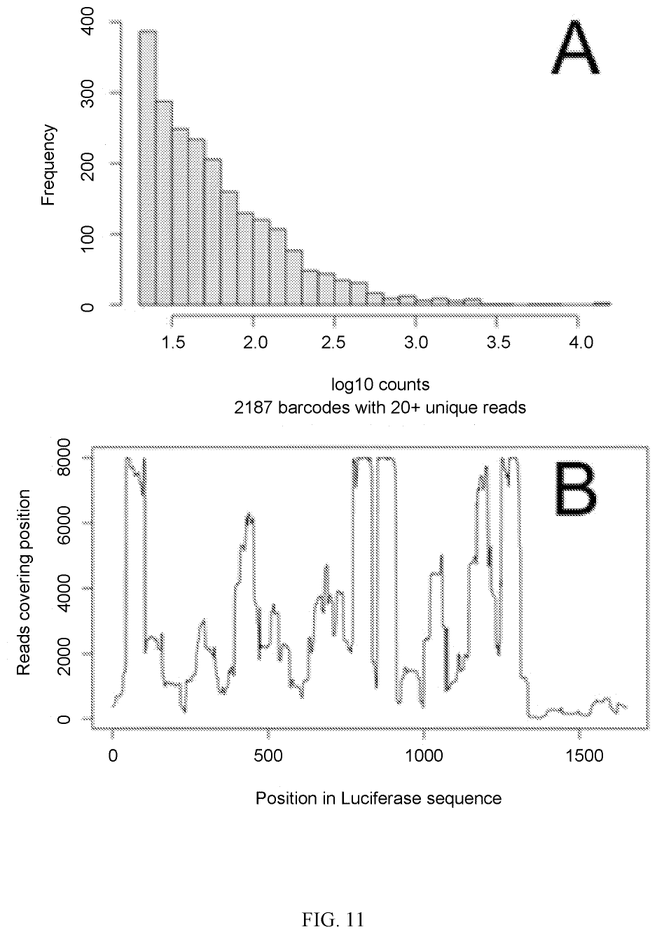

To demonstrate the ability of our approach to synthesize sequenceable cDNA in printed droplets, we printed and subsequently worked up sequencing libraries of purified luciferase mRNA. Sequencing of 3D printed luciferase mRNA using Novoseq 6000 generated 3.3×10⁸ raw reads. The distribution of unique reads per barcode for those exceeded the threshold of 20 reads per barcode indicating that >2,000 barcodes, and over 200,000 unique luciferase reads were captured in total (FIG. 11A). A median of 47 reads were captured per barcode of which 90% of captured reads corresponded to luciferase. A luciferase mRNA coverage plot was made that shows where the captured reads aggregated to examine coverage of the entire luciferase mRNA transcript (FIG. 11B).

To demonstrate the ability of the described technology to perform RNA sequencing on individual EVs, CD9, CD63, and CD81 triple positive human monocyte THP1 cell EVs were purified using Nanopom immunocapture. Twenty thousand droplets were printed with EVs and biochemicals resulting in a predicted ~3,500 droplets that contained one EV and one barcoded gel bead. A trial run was performed on the on the MiSeq platform using a 1M read flowcell. Following RNA sequencing and bioinformatic analysis on our single EV sequencing pipeline (modified inDrop pipeline), 659 individual barcodes were identified after data processing and the corresponding reads were aligned to the genome. The top 10 most abundantly expressed genes were ranked (Table 1).

TABLE 1
Top 10 expressed genes in CD9+/CD63+/CD81+ THP1
EVs as identified by single EV RNA sequencing.

| | Number of barcoded sequences |
|---|---|
| Gene | among single EVs |
| MT-RNR2 | 144 |
| MT-ND4 | 44 |
| MT-CYTB | 26 |
| MALAT1 | 18 |
| MT-COX2 | 12 |
| FOXRED1 | 12 |
| MT-COX1 | 10 |
| NONO | 10 |
| SRSF10 | 10 |
| CLOCK | 9 |

The list includes a number of protein coding, mitochondrial, and noncoding RNAs. Several of the top 10 expressed genes identified by our single EV RNA sequencing have been reported as abundantly expressed in EVs identified by single EV (MALATI, ND4) (Luo T et al. “Transcriptomic Features in a Single Extracellular Vesicle via Single-Cell RNA Sequencing. Small Methods. 2022; 6(11):e2200881) or bulk EV expression analysis (RNR2) (Wang X et al. “Detection of mitochondria-pertinent components in exosomes.” Mitochondrion. 2020; 55:100-10). Under the described conditions, poly (A)⁺ RNA from 659 of 3,500 EVs were sequenced. This corresponds to about one copy of mRNA per 5 EVs, which is comparable to that reported using bulk RNA sequencing (e.g., 1 copy of mRNA per 10 EVs) (Wei Z et al. “Coding and noncoding landscape of extracellular RNA released by human glioma stem cells.” Nat Commun. 2017; 8(1):1145).

The data demonstrate the ability to 3D print individual barcoded hydrogel beads, EVs, and reagents to generate sequenceable libraries. The sequenceable libraries can be used to determine individual droplet barcoded ID's, unique transcript UMI's, and the corresponding cDNA transcripts.

### Example 8. Increasing the Transcriptomic Content of Individual EVs

To increase transcriptomic content of individual EVs sequencing in the EV droplets is performed on the NovaSeq 6000 platform using the S1 flowcell (1.6 B total reads) to sequence all of the RNA contents of individual EVs, including mRNA, miRNA, long noncoding RNAs, tRNA, rRNA as well as their fragments.

### Example 9. Bulk Plasma EV's mtRNA Sequencing

Following the bioinformatics and statistical analysis of the RNA sequencing from a mixture of EVs derived from three different cell lines, we anticipate that UMAP plots may be used to distinguish the three groups of cell line EVs based on their cell type of origin. If this is not possible, then we will move to “pure” samples rather than a mixture. We further anticipate that we will be able to distinguish singlets from multiplets using the analysis described in Aim 2.1 and that the ratios of singlets to multiplets as determined from single EV sequencing parallels those determined using the confocal microscopy imaging of printed fluorescent spheres/EVs (Aim 1.1

1. A method of forming droplets containing a single cell or extracellular vesicle and a single microbead comprising:
forming an aqueous suspension containing cells or extracellular vesicles and microbeads; and
three-dimensional (3D) printing the aqueous suspension into a yield-stress support medium, wherein the 3D printing forms isolated droplets of the aqueous suspension in the yield-stress support medium.

2. The method of claim 1, wherein the extracellular vesicle comprises an exosome or a microvesicle.

3. The method of claim 1 or claim 2, wherein the microbead comprises a DNA barcoded microbead.

4. The method of any one of claims 1-3, wherein the aqueous suspension comprises the extracellular vesicles at a concentration of about 10 to about 60,000 or about 100 to about 500 extracellular vesicles/μL and the microbeads at a concentration of 1900 to about 120,000 microbeads/μL.

5. The method of any one of claims 1-4, wherein greater than 90% of the droplets contain 0, 1, or 2 cells or extracellular vesicles and 0, 1, or 2 microbeads.

6. The method of claim 5, wherein:
(a) ≥65% of droplets contain 1 bead;
(c) ≥25% of the droplets contain 1 cell or extracellular vesicle; or
(d) ≥15% of the droplets contain 1 microbead and 1 cell or extracellular vesicle.

7. The method of any one of claims 1-6, wherein the yield-stress support medium comprises a jammed organic microgel.

8. The method of claim 7, wherein the jammed organic microgel comprises self-assembled block copolymer particles swollen with an organic solvent.

9. The method of any one of claims 1-8, wherein the yield-stress support medium is formed by mixing a mass of one or more block copolymers into a volume of an organic solvent.

10. The method of any one of claims 1-9, wherein the yield-stress support medium is hydrophobic.

11. The method of any one of claims 1-10, wherein the yield-stress support medium is immiscible with the aqueous suspension.

12. The method of any one of claims 1-11, wherein the yield-stress support medium does not comprise a surfactant.

13. The method of any one of claims 1-12, wherein the 3D printing comprises disposing the aqueous suspension through an inner lumen of a printing nozzle into the yield-stress support medium, wherein the printing nozzle is dimensioned and configured to cause formation of the droplet of the aqueous suspension being disposed therethrough.

14. The method of claim 13, wherein the inner lumen is in fluid communication with a supply comprising the aqueous suspension.

15. The method of claim 13 or 14, wherein the printing nozzle has an inner diameter of about 75 μm to about 375 μm.

16. The method of any one of claims 13-15, wherein the aqueous suspension is disposed through the printing nozzle at a flow rate of about 1 to about 300 μL/hr, optionally about 1 to about 135 μL/hr, optionally about 1 to about 30 μL/hr, optionally about 25 μL/hr, or optionally about 3 μL/hr.

17. The method of any one of claims 13-16, wherein the printing nozzle is translated through the yield-stress support medium during the disposing.

18. The method of claim 17, wherein the printing nozzle is translated through the yield-stress support medium at a rate of about 1 to about 30 mm/sec, optionally about 10 mm/sec, or optionally about 20 mm/sec.

19. The method of any one of claims 13-18, wherein the printing nozzle has an inner diameter that is between about 0.1% and about 10% greater than a diameter of the microbead.

20. The method of any one of claims 13-19, wherein an inner diameter of the printing nozzle is between about 0.1% and about 10% larger than an outer diameter of the droplet.

21. The method of any one of claims 1-20, wherein the aqueous suspension further contains one or more reagents to perform a biochemical reaction.

22. The method of claim 21, wherein the biochemical reaction comprises a nucleic acid synthesis reaction, a sequencing reaction, or a nucleic acid amplification reaction, optionally, wherein the nucleic acid amplification reaction comprises PCR or RT-PCR.

23. The method of claim 22, wherein the one or more reagents include one or more of dNTPs, DTT, RNase inhibitor, reaction buffer, DNA polymers, reverse transcriptase, salt, buffer, and surfactant.

24. A three-dimensional (3D) printing apparatus comprising:
a printing nozzle comprising a lumen formed along a longitudinal axis of the printing nozzle and having an inner diameter;
at least one processor; and
at least one memory storing program codes, wherein the at least one memory and the program codes are configured, with the at least one processor, to cause the 3D printing apparatus at least to:
dispose droplets of an aqueous suspension through the printing nozzle into a yield-stress support medium,
wherein respective droplets of said aqueous suspension are disposed in an isolated location in the yield-stress support medium, and
wherein the aqueous suspension contains cells or extracellular vesicles and microbeads.

25. The 3D printing apparatus of claim 24, wherein the one or more extracellular vesicles comprise an exosome or a microvesicle.

26. The method of claim 24 or 25, wherein the microbeads comprise DNA barcoded microbeads.

27. The 3D printing apparatus of any one of claims 24-26, wherein the yield-stress support medium comprises a jammed organic microgel.

28. The 3D printing apparatus of claim 27, wherein the jammed organic microgel comprises self-assembled block copolymer particles swollen with an organic solvent.

29. The 3D printing apparatus of claim 28, wherein the yield-stress support medium is immiscible with the aqueous suspension.

30. The 3D printing apparatus of any one of claims 24-29, further comprising: a mixer configured to mix a mass of one or more block copolymers with a volume of an organic solvent to form the yield-stress support medium.

31. The 3D printing apparatus of any one of claims 24-30, further comprising:
a reservoir configured to store a supply of the aqueous suspension,
wherein the at least one memory and the program codes are further configured, with the at least one processor, to cause the 3D printing apparatus at least to:
communicate a portion of the aqueous suspension from the supply through the inner lumen of the printing nozzle, the printing nozzle being dimensioned and configured to cause formation of the droplet of the aqueous suspension being communicated therethrough.

32. The 3D printing apparatus of claim 31, further comprising: a conduit configured to communicate said one or more extracellular vesicles into the printing nozzle.

33. The 3D printing apparatus of claim 32, wherein the conduit is dimensioned and configured to communicate exactly one exosome into the barcoded hydrogel microsphere once disposed within the droplet and before the droplet is disposed within the yield-stress support material.

34. The 3D printing apparatus of claim 31, wherein an inner diameter of the printing nozzle is between about 0.1% and about 10% larger than an outer diameter of the droplet.

35. A method of performing a biochemical reaction on the contents of a single cell or extracellular vesicle comprising:
forming one or more droplets as in any one of claims 1-23 and performing the biochemical reaction within the one or more droplets.

36. The method of claim 35, further comprising: lysing the cell or extracellular vesicle within the droplet.

37. The method of claim 36, wherein the extracellular vesicle comprises an exosome.

38. The method of claim 37, wherein lysing the cell or extracellular vesicle comprises heating the yield-stress support medium containing the droplets to about 50° C.

39. The method of any one of claims 35-38, further comprising: separating the yield-stress support medium and the droplets.

40. The method of claim 39, wherein separating the yield-stress support medium and droplets comprises centrifuging the yield-stress support medium and the droplets contained in the yield-stress support medium.

41. The method of claim 40, wherein centrifuging the yield-stress support medium containing the droplets does not result in fusion of the droplets.

42. The method of any one of claims 35-41, wherein the method further comprises pooling the droplets.

43. The method of any one of claim 35-42, wherein the microbead comprises a DNA barcoded microbead.

44. The method of claim 43, wherein the biochemical reaction comprises a DNA synthesis reaction and/or a DNA sequencing reaction.

45. The method of claim 44, wherein the method further comprises exposing the droplets to 350 nm light thereby releasing barcoded cDNA primers from the microbeads.

46. The method claim 45, wherein the method further comprises performing a DNA amplification reaction.

47. The method of claim 45 or 46, wherein the DNA sequencing reaction is used to sequence one or more RNAs of an extracellular vesicle.

48. The method claim 47, wherein the biochemical reaction further comprises adding a poly A tail to at least one RNA present in the extracellular vesicle.

49. The method of claim 47, wherein the biochemical reaction does not comprise adding a poly A tail to any RNAs present in the extracellular vesicle.

50. A method of sequencing one or more RNAs present in a plurality of individual exosomes comprising:
(a) forming a plurality of droplets as in any one of claims 3-23;
(b) heating the yield-stress support medium containing the droplets to about 50° C., thereby lysing the exosomes;
(c) exposing the droplets to 350 nm light, thereby releasing barcoded cDNA primers from the microbeads;
(d) performing a reverse transcriptase reaction, wherein reverse transcriptase present in the droplet reverse transcribes the one or more RNAs present in the exosomes using primers released from the DNA-barcoding microbeads thereby forming cDNAs;
(e) centrifuging the yield-stress support medium and the droplets contained in the yield-stress support medium, thereby separating the yield-stress support medium from the droplets and collecting the droplets;
(f) performing a sequencing reaction on the cDNAs.

51. The method of claim 50, further comprising performing a DNA amplification reaction prior to step (f), thereby amplifying the cDNAs of step (d).

52. The method of any one of claims 47-51, where the one or more the RNAs are selected from the group consisting of: mRNA, non-coding RNA, Y RNA, lincRNA, miRNA, tRNA, rRNA, and fragments or combinations thereof.

53. The method of claim 50 or 51, further comprising adding a poly(A) tail to one or more of the RNAs prior to step (d).

54. A method of analyzing a sample from a subject comprising: sequencing RNAs from extracellular vesicles as in any of claims 47-53, thereby identifying RNAs expressed by one or more cell types in the subject.

55. The method of claim 54, wherein identifying RNAs expressed by one or more cell types in the subject is used to diagnose a disease.
